(19) United States
(12) Patent Application Publication
(10) Pub. No.: US 2026/0290545 A1
(43) Pub. Date: Sep. 24, 2026

(54) SYSTEM

(71) Applicant: SoftBank Group Corp., Tokyo (JP)

(72) Inventor: Masahiro OKUI, Tokyo (JP)

(73) Assignee: SoftBank Group Corp., Tokyo (JP)

(21) Appl. No.: 19/567,259

(22) Filed: Mar. 16, 2026

(30) Foreign Application Priority Data
Mar. 19, 2025 (JP) 2025-045043

(51) Int. Cl. G16H 20/60; G06N 3/0475; G06V 10/764; G06V 10/77

(52) U.S. Cl. CPC G16H 20/60; G06N 3/0475; G06V 10/764; G06V 10/7715; G06V 2201/07

(57) ABSTRACT

A system includes a processor that is configured to acquire an image of an interior of a refrigerator and identify stored food ingredients by performing image analysis on the image, provide, to a user, an interface for inputting at least a time available for cooking and a date and time available for shopping, generate a prompt text to be input to a generative artificial intelligence model based on the acquired image and inputted information, and cause the generative artificial intelligence model to propose an optimal meal plan based on the prompt text.

### CROSS-REFERENCE TO RELATED APPLICATION

This application is based on and claims priority under 35 USC 119 from Japanese Patent Application No. 2025-045043 filed on Mar. 19, 2025, the disclosure of which is incorporated by reference herein.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

Conventional meal planning and grocery purchasing processes impose a significant cognitive and time burden on users. A user typically must manually inspect the interior of a refrigerator, remember or record available ingredients, search for recipes that effectively utilize those ingredients, and align such recipes with personal constraints including time available for cooking and dates and times at which the user can go shopping or receive deliveries. Furthermore, when additional ingredients are needed, the user must individually compare prices and inventory across retailers, determine which items can be purchased within a given budget, and manually execute online or in-store purchase procedures. Additionally, the user must separately arrange delivery or pickup timing such that purchased ingredients arrive in advance of planned cooking times. As a result, existing approaches do not sufficiently reduce the overall burden of menu planning, ingredient selection, budget-based purchasing, and delivery scheduling. There is a need for a system that automatically recognizes food ingredients stored in a refrigerator, generates an optimal meal plan tailored to the user's time and shopping constraints, automatically proposes ingredients purchasable within a specified budget using network-based information, performs purchase procedures, and coordinates delivery so that the necessary ingredients are delivered at a user-specified date and time.

### SUMMARY

In order to solve the foregoing problems, according to one aspect of the present invention, there is provided a system comprising a processor, wherein the processor is configured to acquire an image of an interior of a refrigerator and identify stored food ingredients by performing image analysis on the image, provide to a user an interface for inputting at least a time available for cooking and a date and time available for shopping, generate a prompt text to be input to a generative artificial intelligence model based on the acquired image and the inputted information, and cause the generative artificial intelligence model to propose an optimal meal plan based on the prompt text. In addition, in some embodiments, the processor is further configured to obtain, via a network, price information and inventory information for food ingredients, automatically propose purchasable food ingredients based on a budget specified by the user, and perform a purchase procedure for the proposed food ingredients. In further embodiments, the processor is configured to coordinate with a delivery service to perform a delivery procedure so that the proposed food ingredients are delivered at a date and time specified by the user. By integrating refrigerator image analysis, user constraint input, generative artificial intelligence-based meal planning, budget-constrained ingredient proposal using network-acquired price and inventory information, automated purchase processing, and delivery coordination into a unified system, the invention reduces the user's burden associated with menu planning, ingredient selection and procurement, and delivery scheduling.

The term “system” refers to an apparatus or combination of hardware and software components that collectively execute the processing described in the claims.

The term “processor” refers to one or more hardware processing units, such as a central processing unit (CPU), graphics processing unit (GPU), or dedicated accelerator, that execute instructions to perform the functions recited in the claims.

The term “image of an interior of a refrigerator” refers to digital image data representing at least a portion of a storage space inside a refrigerator in which food ingredients are stored.

The term “image analysis” refers to processing of digital image data using algorithmic or machine-learning techniques, including object detection or classification, to detect and identify items present in the image.

The term “food ingredients” refers to edible items, including raw or processed foods, that can be used, singly or in combination, to prepare meals.

The term “identify stored food ingredients” refers to determining, from the image of the interior of the refrigerator, one or more types of food ingredients that are present in the refrigerator.

The term “interface” refers to a graphical user interface, voice interface, or other input/output mechanism through which a user can provide information to, and receive information from, the system.

The term “time available for cooking” refers to one or more time periods or points in time during which the user is able or intends to perform cooking operations.

The term “date and time available for shopping” refers to one or more dates and associated time periods during which the user can perform shopping or receive purchased items, including delivery time windows.

The term “prompt text” refers to a textual or text-encoded data structure that expresses a request, condition, or context to be provided as input to a generative artificial intelligence model.

The term “generative artificial intelligence model” refers to a machine-learning model, such as a large language model or multimodal generative model, that generates output data, including text or structured information, in response to an input such as a prompt text.

The term “optimal meal plan” refers to a set of one or more proposed meals, menus, or recipes that are determined in view of at least the identified food ingredients and the user's inputted time and shopping constraints, and that are evaluated according to one or more criteria, such as ingredient utilization, convenience, or user preference.

The term “price information” refers to data representing monetary prices or costs associated with purchasing particular food ingredients or products.

The term “inventory information” refers to data indicating availability, stock levels, or out-of-stock status of particular food ingredients or products at one or more retailers or suppliers.

The term “budget specified by the user” refers to a monetary limit input by the user representing a maximum allowable total cost for purchasing proposed food ingredients.

The term “purchasable food ingredients” refers to food ingredients or corresponding products that are available for purchase, as determined from price information and inventory information, and that are selectable for ordering within the user's specified budget.

The term “purchase procedure” refers to a sequence of operations for ordering products, including generating an order, transmitting order details to a retailer or service via a network, and receiving an order confirmation.

The term “delivery service” refers to a third-party or affiliated logistics provider, retailer, or system that transports purchased food ingredients from a source location to a destination specified by the user.

The term “delivery procedure” refers to operations for arranging, scheduling, or confirming the transportation and delivery of purchased food ingredients, including specifying delivery date and time and communicating such information to the delivery service.

The term “date and time specified by the user” refers to a calendar date and an associated time or time window that the user inputs as a desired delivery period for receiving proposed food ingredients.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

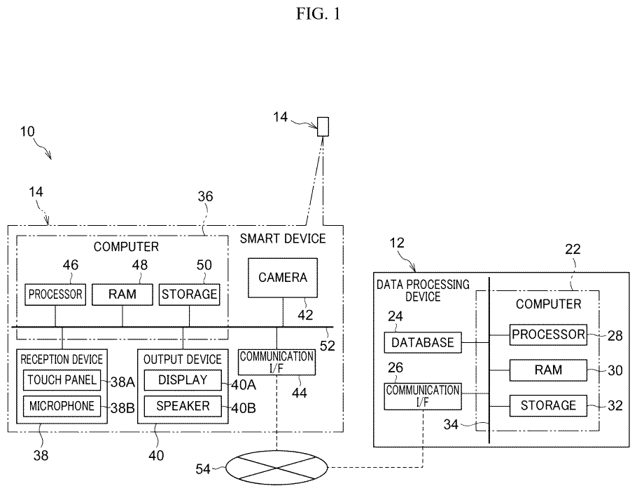

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

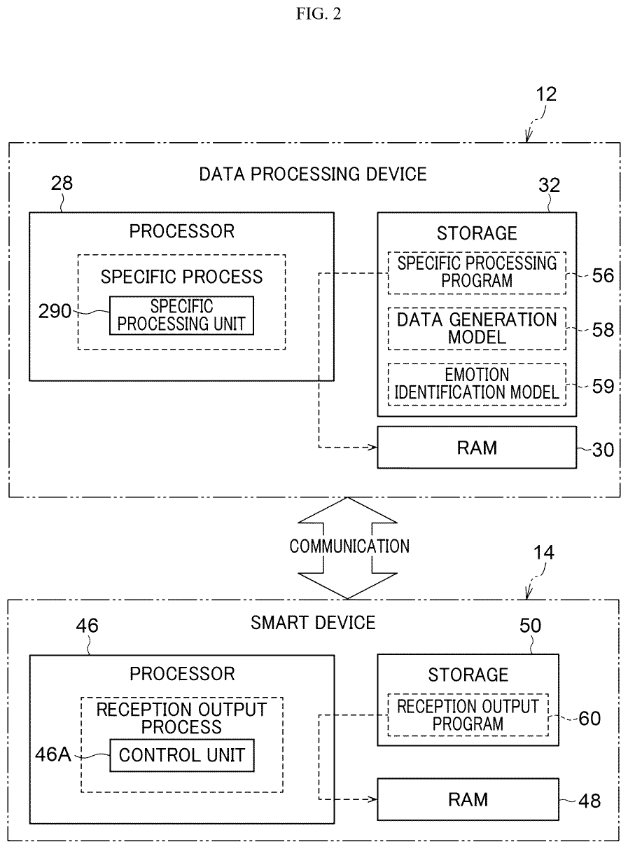

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60.

The reception and output processing is implemented by the processor 46 operating as the control unit 46A according to the reception and output program 60 executed in the RAM 48.

Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58.

The data processing device 12 may be a server device, and may be a terminal device owned by the user (for example, a mobile phone, a robot, a home electrical appliance, or the like).

Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

Conventional computer-implemented meal planning systems are limited in their ability to leverage heterogeneous data sources, such as image data of a storage apparatus interior, user-specific temporal constraints, and external commerce and logistics information, in a unified and machine-understandable form. Typical systems either rely on users to manually enter ingredient data and time constraints, or apply rule-based recommendation engines that are not capable of flexibly adapting to changing user contexts and inventory conditions. As a result, conventional systems impose a high interaction burden on the user, are prone to input errors and omissions, and often generate meal suggestions that do not reflect actual ingredient availability or realistic preparation schedules.

Furthermore, existing systems generally treat image recognition, user interface interaction, recommendation logic, and commerce or delivery coordination as loosely coupled components. This fragmented architecture prevents the computation platform from performing end-to-end optimization, causes redundant data transformations, and degrades performance and reliability. In particular, conventional systems do not provide an efficient computational mechanism for transforming low-level machine data (such as detected items, time values, and budget constraints) into a structured representation that can be directly consumed by a generative AI model through a well-formed prompt sentence. This leads to suboptimal utilization of generative models and reduces the accuracy, consistency, and usefulness of generated plans.

In addition, known systems often fail to exploit the generative capabilities of modern AI models to dynamically synthesize meal plans that integrate inventory usage, user schedule, purchasing options, and delivery timing in a coordinated manner. Instead, they typically perform separate processes for recommendation, purchasing, and delivery, which increases processing latency and complicates error handling across multiple services. Such an approach does not substantially improve the underlying computer technology, because it does not provide a technical framework for automatic acquisition, normalization, and orchestration of multi-source data into a single computational pipeline driven by a generative AI model.

Therefore, there is a need for a computer-implemented system and method that improve the functioning of the underlying computing environment by: (i) automatically acquiring image data from a storage apparatus and transforming it into structured item information through image analysis; (ii) integrating user time constraints, budget information, and external commerce and delivery data into a coherent internal representation; and (iii) generating, via a generative AI model using a carefully constructed prompt sentence, a machine-usable meal plan that is tightly coupled with purchase and delivery processing. Such a system should reduce manual user interaction, improve data consistency and processing efficiency, and enable more accurate and context-aware meal planning, while enhancing the technical operation of servers, databases, and AI inference components that implement the solution.

The specific processing by the specific processing unit 290 of the data processing device 12 in Example 1 is realized by the following means.

The present invention provides a server comprising a processor configured to acquire image data from an image acquisition apparatus that captures an interior of a storage apparatus, process the image data based on image analysis to specify item information stored in the storage apparatus, provide, on a terminal, an information input/output interface through which a user inputs time information for cooking and time information for purchasing items, receive the time information from the terminal via a communication network, store the time information in a storage device, generate structured data including the specified item information and the time information, generate a prompt sentence in natural language based on the structured data, input the prompt sentence into a generative AI model, generate meal plan information including a plurality of cooking menus and an execution time corresponding to each of the cooking menus based on natural language generation processing performed by the generative AI model, convert the generated meal plan information into display data for the terminal, transmit the display data to the terminal via the communication network, acquire price information and inventory information of items from an electronic commerce platform via the communication network, generate a further prompt sentence for input to the generative AI model based on the acquired item information, the time information, and budget information, obtain, from the generative AI model, additional meal plan information including candidate purchase items and cooking menus using the candidate purchase items, automatically select candidate purchase items that conform to the budget information, transmit purchase processing information regarding the selected candidate purchase items to the electronic commerce platform, determine a desired delivery time based on an execution time of the cooking menus included in the meal plan information and the purchase processing information, and transmit delivery request information including the desired delivery time and the candidate purchase items to a delivery management platform so that the candidate purchase items are delivered at a time specified by the user or at a time determined based on the meal plan information. This enables an integrated and computer-implemented processing pipeline in which multi-modal input data are automatically normalized into structured data, transformed into an optimized prompt sentence for a generative AI model, and used to compute meal plans, purchase operations, and delivery schedules in a coordinated manner, thereby reducing user input burden, improving the efficiency and reliability of server-side computations, and enhancing the technical performance of the underlying computing system.

The term “system” refers to a combination of hardware and software components, including at least one processor, memory, communication interfaces, and associated programs, that cooperate to execute the claimed functions.

The term “processor” refers to a hardware processing unit, such as a central processing unit or a microcontroller, that executes machine-readable instructions to perform logical and arithmetic operations required by the claimed system.

The term “storage apparatus” refers to a physical apparatus configured to store items under controlled environmental conditions, such as a refrigeration apparatus or a freezing apparatus, in which perishable or non-perishable goods are kept.

The term “image acquisition apparatus” refers to an electronic apparatus capable of capturing image data of a scene, such as a camera, an image sensor module, or a multifunction terminal equipped with an imaging function.

The term “image data” refers to digital data representing a captured image or sequence of images, encoded in a predetermined format, and processable by a computing device using image analysis techniques.

The term “image analysis” refers to computer-implemented processing applied to image data, including at least one of object detection, object recognition, classification, segmentation, and feature extraction, to derive semantic information from the image data.

The term “item information” refers to structured data representing items stored in the storage apparatus, including at least one of an item type, an item identifier, a quantity, a storage location, and an acquisition or detection time.

The term “terminal” refers to an electronic device, such as a mobile communication device, a tablet computing device, or a personal computing device, that provides a user interface and is configured to communicate with the server via a communication network.

The term “information input/output interface” refers to a software-implemented user interface presented on a terminal, including graphical elements, control elements, and data fields, through which a user can input data and receive output data.

The term “time information” refers to data indicating a time or time period, including at least one of a specific date and time, a time window, a recurrence pattern, or a duration associated with cooking or purchasing activities.

The term “communication network” refers to a wired or wireless data communication infrastructure, such as a local area network, a wide area network, or a public communication network, over which digital data are exchanged between the server, terminals, and external platforms.

The term “storage device” refers to a computer-readable memory device, such as a magnetic storage device, a solid-state storage device, or a non-volatile memory, configured to store data used by the processor.

The term “structured data” refers to data arranged in a predefined format, such as a record, table, object, or document with defined fields, that enables systematic processing by a computing device.

The term “prompt sentence” refers to a natural language text, or a sequence of natural language texts, constructed according to a predetermined scheme and provided as input to a generative AI model to specify a desired computational or generative task.

The term “natural language” refers to a human language, such as English or another human language, expressed as text that can be processed by a natural language processing system.

The term “generative AI model” refers to a machine learning model configured to generate output data, such as natural language text, in response to input data, the model having been trained using artificial intelligence techniques including at least one of deep learning and probabilistic modeling.

The term “natural language generation processing” refers to computer-executed processing performed by a generative AI model to output natural language text based on an input prompt sentence and associated internal model parameters.

The term “meal plan information” refers to structured or semi-structured data representing a plan for meals, including at least one of dish names, ingredient usage, preparation steps, execution times, and scheduling information.

The term “cooking menu” refers to data specifying at least one dish or recipe, including an identification of a prepared food item and optionally associated ingredients, instructions, and preparation time.

The term “execution time” refers to time information associated with a cooking menu, including at least one of a start time, an end time, or a recommended time for preparing or consuming the meal.

The term “display data” refers to data formatted for presentation on a display apparatus of a terminal, including at least one of text, icons, images, and layouts derived from underlying structured data.

The term “electronic commerce platform” refers to a computer-implemented service accessible via a communication network, configured to manage product listings, pricing data, inventory data, and purchase transactions for goods or services.

The term “price information” refers to data indicating a monetary value associated with an item, including at least one of a unit price, a discount, a tax amount, or a total price for a specified quantity.

The term “inventory information” refers to data indicating availability of items, including at least one of stock quantity, stock status, replenishment timing, or location information in a supply chain.

The term “budget information” refers to data representing a monetary constraint or range specified by or determined for a user, including a maximum allowable expenditure for purchasing items.

The term “candidate purchase items” refers to items identified by the system as potential items to be obtained through a purchase operation, based on at least one of meal plan information, price information, inventory information, and budget information.

The term “purchase processing information” refers to data used to execute or request a purchase transaction for items, including at least one of item identifiers, quantities, payment information, user identifiers, and delivery destination information.

The term “delivery management platform” refers to a computer-implemented service that manages logistics operations, including reception of delivery requests, scheduling of shipments, and coordination of carriers to deliver items to specified destinations.

The term “desired delivery time” refers to time information specifying when items are requested to be delivered, the time being determined based on at least one of a cooking schedule, purchase processing timing, or a user preference.

The term “delivery request information” refers to data transmitted to a delivery management platform to request delivery of items, including at least one of item identifiers, quantities, destination information, and desired delivery time.

In one embodiment, a server cooperates with at least one terminal and at least one image acquisition apparatus to implement the claimed system. The server includes a processor, a main memory, a non-volatile storage device, and a network interface that are interconnected by a hardware bus. The terminal includes a processor, a display, an input device such as a touch panel, a local storage, and a wireless communication interface. The image acquisition apparatus includes an image sensor and an optical system and may be integrated into the terminal or implemented as a separate device that is communicatively coupled to the terminal.

The server executes server-side application software implemented, for example, using a web application framework. The server uses an operating system, such as a general-purpose server operating system, and executes software modules for request handling, image analysis, database access, and generative AI inference. The server accesses a database management system, such as a relational database, to store and retrieve item information, user profiles, time information, meal plan information, and transaction logs. The terminal executes a dedicated client application developed, for example, using a mobile application framework, which provides a graphical user interface to the user and communicates with the server over a communication network using a communication protocol such as HTTPS.

The terminal uses the image acquisition apparatus to capture image data of an interior of a storage apparatus, such as a refrigeration apparatus. The terminal stores the captured image data into local storage as a file encoded in a format such as JPEG or PNG. The terminal then transmits the image data to the server via the communication network as part of a request message. The server receives the request message through the network interface and passes the image data to an image analysis module.

The server applies image analysis processing to the received image data to specify item information stored in the storage apparatus. The server may use an image recognition library or a cloud-based image analysis service to perform object detection and classification. In one embodiment, the server uses a convolutional neural network (CNN) model trained on labeled images of common food items. The CNN model includes multiple convolutional layers, pooling layers, and fully connected layers. The server inputs the image data as a tensor to the CNN, performs forward propagation in which each convolutional layer applies learned kernels to local regions of the image, and obtains class probability scores for candidate item categories.

The server uses a softmax output layer to generate class probabilities and selects item categories with probabilities higher than a predetermined threshold. The server includes a mapping module that maps the selected categories to internal item identifiers and generates item information that includes, for each item, at least an item identifier, a confidence score, and a detection timestamp. The server stores the item information in the database as structured records. The server optionally aggregates item information across multiple images by performing record merging based on category similarity and temporal proximity, thereby improving robustness against occlusions and partial views in individual images.

The terminal provides an information input/output interface to the user. The terminal displays input fields for time information for cooking and time information for purchasing items. The terminal may use graphical controls such as date pickers and time pickers to reduce input errors. The user inputs, for example, a desired dinner time for each day of a week and one or more time windows during which the user can visit a store or receive a delivery. The terminal validates the input locally by checking a logical order of start times and end times and by normalizing the time information to a standardized representation, such as coordinated universal time in ISO 8601 format. The terminal then transmits the time information to the server.

The server receives and stores the time information in association with the item information and the user identifier. The server generates structured data that combines the item information and the time information. In one embodiment, the server constructs a data structure that includes a list of items, each with an item identifier and a quantity estimate, and a schedule structure that represents one or more target cooking times and shopping time windows. The server may further include budget information, dietary preferences, and previously recorded user feedback as additional fields in the structured data.

The server constructs a prompt sentence in natural language based on the structured data. The server uses a prompt construction module that applies a template-based algorithm. The module inserts the item names, time constraints, and other parameters into predetermined sentence patterns to form a coherent instruction for a generative AI model. For example, the server may generate the following prompt sentence:

“The ingredients currently available in the refrigerator are: tomatoes, cheese, and lettuce. I want to cook dinner today at 18:00. I can go grocery shopping tomorrow morning between 9:00 and 11:00. Using a generative AI model, please create a one-week meal plan that efficiently uses these ingredients and suggests 3 different dinner menus. For each menu, provide a dish name, a short recipe, required ingredients, and an estimated cooking time.”

The server uses a generative AI model to process the prompt sentence. In one embodiment, the generative AI model is a large-scale neural network based on a transformer architecture.

The transformer includes an embedding layer that maps input tokens of the prompt sentence into continuous vector representations, multiple self-attention layers that compute attention weights between tokens to capture long-range dependencies, and feed-forward layers that transform the attended representations. The model parameters include weight matrices for query, key, and value projections in the attention mechanism, as well as weights and biases for feed-forward layers. The server stores the model parameters in memory and uses them to perform inference.

The server tokenizes the prompt sentence using a subword tokenization algorithm and inputs the resulting token sequence to the generative AI model. The server performs autoregressive generation in which the model computes, for each time step, a probability distribution over possible next tokens using a softmax function applied to the output of the final transformer layer. The server selects a next token according to a sampling policy that may include a temperature parameter and a top-k or top-p selection. The server iteratively appends generated tokens to the sequence until an end-of-sequence condition is met. In this way, the generative AI model outputs text describing a meal plan, including dish names, descriptions, ingredient usage, and schedule information.

The server post-processes the generated text to convert it into structured meal plan information. The server may instruct the generative AI model, via the prompt sentence, to output in a semi-structured list format to facilitate parsing. The server applies parsing algorithms, such as regular expression matching or rule-based segmentation, to identify individual cooking menus, associated ingredients, and execution times. The server normalizes ingredient names using the same mapping table used for item recognition, and verifies that the generated menus do not require unavailable items unless purchasing is allowed. The server stores the finalized meal plan information in the database.

The terminal receives display data derived from the meal plan information. The server converts the meal plan information into a display-oriented representation that groups menus by day and time and associates icons or images with each dish. The terminal renders a calendar-like view or a list view showing, for example, “Tomato and Cheese Salad” for today at 18:00 and “Lettuce and Cheese Sandwich” for another day. The user can select each menu to view detailed preparation steps and required ingredients.

The server also acquires price information and inventory information of items from an electronic commerce platform. The server issues API requests to the electronic commerce platform, specifying item identifiers and, optionally, store identifiers or regions. The electronic commerce platform returns structured data that includes unit prices and stock status. The server incorporates this information into the structured data and constructs a further prompt sentence that explicitly requests menu planning with purchasing options and budget constraints. The server may generate a prompt sentence such as:

“Available ingredients in the refrigerator are tomatoes, cheese, and lettuce. The user wants to cook dinner at 18:00 today and can go grocery shopping tomorrow morning between 9:00 and 11:00. The user has a budget of 30 units of currency for additional ingredients. Based on the current prices and inventory of items, propose 3 dinner menus for this week. For each menu, specify which existing ingredients will be used, which additional ingredients should be purchased within the budget, and the estimated cooking time.”

The server inputs this prompt sentence into the generative AI model and obtains additional meal plan information that includes candidate purchase items. The server compares the suggested purchase items with the budget information and applies deterministic selection algorithms, such as greedy selection or knapsack-type optimization, to ensure that total cost does not exceed the budget. The server then generates purchase processing information and transmits it to the electronic commerce platform to initiate or schedule a purchase transaction.

The server determines a desired delivery time by correlating the execution times of cooking menus in the meal plan information with available delivery slots obtained from a delivery management platform. The server generates delivery request information that includes item identifiers, quantities, delivery address, and a desired delivery time that precedes the related cooking execution time by a margin sufficient for preparation. The server transmits the delivery request information to the delivery management platform, which arranges physical delivery of the items.

This configuration improves computer technology in several ways. The server reduces communication overhead and processing latency by transforming heterogeneous data sources (image data, user time inputs, pricing data, and inventory data) into structured data within a unified internal schema, so that the generative AI model can receive a compact and information-dense prompt sentence instead of raw or partially processed data. Because the generative AI model is driven by a carefully constructed prompt sentence reflecting normalized, pre-filtered data, the server reduces redundant computations at the inference stage and reduces the need for repeated queries to external services.

The server improves accuracy of meal planning by applying neural network-based image analysis and transformer-based generative modeling in a coordinated fashion. The image analysis CNN reduces errors in item detection through learned feature extraction, as opposed to manual tagging, which is prone to user oversight. The generative AI model constructs menus and schedules that take into account complex constraints that would be difficult to encode in conventional rule-based systems, such as overlapping time windows, partial ingredient usage, and budget constraints combined with inventory availability.

From a computational standpoint, the server improves data management by organizing all relevant data into indexed tables or documents with explicit relationships, enabling optimized queries and cache strategies. The server may maintain a cache of frequently accessed ingredient and price information to reduce the number of external API calls, thereby reducing communication latency and bandwidth usage. The server may also apply batching of prompt sentences for multiple users to a shared generative AI inference engine, improving throughput and hardware utilization.

The generative AI model operates with rules and internal representations that differ fundamentally from simple human decision-making or rule-based automation. The transformer architecture computes self-attention scores across the entire token sequence, allowing it to capture dependencies between ingredients, times, and budgets that are not represented as explicit rules but as learned patterns in the model parameters. During training, the model minimizes a loss function, such as cross-entropy loss, over large corpora of cooking-related and planning-related text, and updates its weights using gradient-based optimization. The server may further fine-tune the generative AI model using domain-specific training data that include examples of meal plans conditioned on ingredient lists and time constraints. The server may apply data augmentation techniques such as paraphrasing and time-shift augmentation to increase diversity of training samples and improve model robustness.

The server uses this trained generative AI model to produce outputs that are more consistent and comprehensive than those based on manually programmed rules, because the model can generalize to novel combinations of ingredients and constraints. The server's prompt construction and post-processing modules ensure that the generative AI model is integrated into an end-to-end computational pipeline that is deterministic at the system level, even though the generative model itself is probabilistic. This integration yields technical effects such as reduced error rates in planning, improved speed of generating viable meal plans, and an overall improvement in the efficiency of server-side resource usage.

Alternative embodiments are possible. The server may host the generative AI model locally, using specialized hardware such as graphics processing units or tensor processing units, or may access an external AI inference service over the communication network. The image analysis may be performed either entirely on the server, entirely on the terminal, or in a distributed manner where the terminal performs initial preprocessing and the server performs final classification. The structured data format may be implemented using different data models, such as relational schemas or document-based schemas, as long as item information and time information remain explicitly representable.

In another embodiment, the server supports multiple storage apparatuses associated with different rooms or locations, and the prompt sentence construction includes location attributes to distinguish items stored in different storage apparatuses. In yet another embodiment, the server incorporates feedback from the user regarding the acceptability of generated menus, and uses such feedback to update model parameters or adjust prompt templates, thereby improving personalization over time. In all of these embodiments, the server, the terminal, and the generative AI model cooperate to implement specific technical data structures and processing flows that improve computational performance, data accuracy, and system-level efficiency, beyond what would be achieved by merely automating a human planning process.

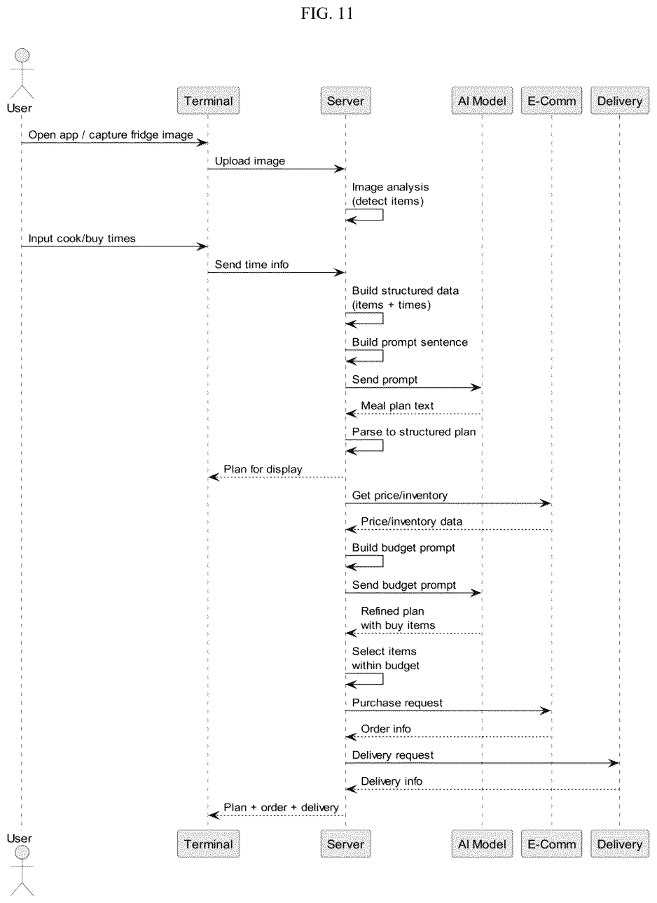

The following describes the processing flow using FIG. 11.

### Step 1:

User operates the terminal to capture an image of the interior of a storage apparatus.

Input: A real-world scene inside the storage apparatus.

Output: Digital image data stored on the terminal.

User launches a dedicated application on the terminal and selects a function for photographing the storage apparatus interior. User points the terminal's camera toward the interior and taps a capture button. Terminal uses a camera API to control the image sensor, converts analog light signals into digital pixel data, and encodes the captured frame as a compressed image file, such as a JPEG or PNG file, which is stored in a local file system of the terminal.

### Step 2:

Terminal transmits the captured image data to the server.

Input: Image file stored on the terminal.

Output: Image data received and stored by the server.

Terminal reads the image file from local storage, encapsulates the binary data into a request message, for example using an HTTPS POST with multipart/form-data encoding, and adds metadata such as user identifier and timestamp. Terminal sends the request over a communication network. Server receives the request through a network interface, validates the message (e.g., checks authentication token and file type), and writes the received image data to a storage device, optionally assigning a unique image identifier.

### Step 3:

Server performs image analysis to extract item information from the image data.

Input: Stored image data associated with the storage apparatus.

Output: Structured item information representing items detected in the storage apparatus.

Server loads the image data into an image analysis module and resizes and normalizes pixel values to a fixed input size for a convolutional neural network. Server feeds the normalized image tensor into the network, which applies convolution operations, non-linear activations, and pooling operations to extract hierarchical features. Server computes class probabilities using a softmax layer and selects item classes exceeding a confidence threshold. Server converts each detected class into an internal item identifier using a mapping table, and constructs item records including item identifier, confidence score, and detection timestamp.

Server stores these item records into a database.

### Step 4:

Terminal collects time information for cooking and purchasing from the user.

Input: User's intended schedule and constraints.

Output: Normalized time information stored on the terminal.

User views an input screen provided by the terminal that includes date and time selectors for cooking times and purchasing or delivery time windows. User selects, for example, a cooking time of 18:00 on a particular date and a purchasing window on the following morning.

Terminal validates that each time range has a valid start and end and converts the selected values into a standardized time format, such as ISO 8601 strings, forming a time information data structure that includes cooking times and purchasing windows.

### Step 5:

Terminal transmits the time information to the server.

Input: Normalized time information data structure on the terminal.

Output: Time information stored in the server's database.

Terminal encapsulates the time information in a request body, for example as JSON, and sends an HTTPS request to a server endpoint, attaching the user identifier for association.

Server receives the request, verifies the user's credentials, and parses the JSON content to extract cooking times and purchasing or delivery windows. Server writes the extracted time information into a database, linking each record to the corresponding user and to the item information previously stored.

### Step 6:

Server combines item information and time information into structured data.

Input: Item records from image analysis and time records from user input.

Output: Unified structured data representing the current planning context.

Server queries the database to retrieve the latest item records for a user and associated time records. Server merges these records into a composite data structure that includes a list of item identifiers, optional quantity estimates, and one or more time constraints for cooking and purchasing. Server may also add auxiliary fields, such as user dietary preferences or budget if available. This composite structure forms the basis for generating a context-rich prompt sentence.

### Step 7:

Server constructs a prompt sentence in natural language for the generative AI model.

Input: Unified structured data containing item information and time information.

Output: A natural language prompt sentence text.

Server passes the unified structured data to a prompt construction module. The module inserts item names and time values into predefined templates, orders the information according to a logical pattern (for example: ingredients, cooking time, purchasing window, and requested output format), and concatenates them into a coherent text. Through this data transformation, server converts numeric and symbolic data into descriptive text. For example, server may generate the prompt sentence:

“The ingredients currently available in the refrigerator are: tomatoes, cheese, and lettuce. I want to cook dinner today at 18:00. I can go grocery shopping tomorrow morning between 9:00 and 11:00. Using a generative AI model, please create a one-week meal plan that efficiently uses these ingredients and suggests 3 different dinner menus. For each menu, provide a dish name, a short recipe, required ingredients, and an estimated cooking time.”

### Step 8:

Server generates meal plan information by processing the prompt sentence with the generative AI model.

Input: Natural language prompt sentence.

Output: Generated text describing one or more cooking menus and schedules.

Server tokenizes the prompt sentence into tokens using a subword tokenizer and converts each token to an embedding vector. Server inputs the embedded sequence into a transformer-based generative AI model. For each generation step, the model computes self-attention scores between tokens, multiplies them by learned weight matrices, and passes the resulting hidden states through feed-forward layers. Server obtains a probability distribution over possible next tokens from a softmax layer and selects tokens according to a sampling strategy until a termination token appears. This iterative computation yields a generated text that may contain menu names, ingredient usage, preparation steps, and suggested times.

### Step 9:

Server converts the generated text into structured meal plan information.

Input: Generated natural language text from the generative AI model.

Output: Structured meal plan records stored in the database.

Server applies parsing logic to the generated text, such as splitting the text at delimiters or headings corresponding to individual menus. Server extracts, for each menu, the dish name, list of ingredients, and associated execution time by analyzing text patterns. Server maps ingredient names back to internal item identifiers, normalizes time expressions into standardized time formats, and forms structured meal plan entries. Server inserts these entries into the database and associates them with the user identifier and planning period.

### Step 10:

Server acquires external price and inventory information for potential purchase items.

Input: Internal list of items to be considered for purchase.

Output: Price information and inventory information for those items.

Server selects items from the meal plan that are not currently available in sufficient quantity in the storage apparatus. Server constructs query messages including item identifiers and optionally location parameters, and sends them to an electronic commerce platform via its application programming interface. The electronic commerce platform returns structured responses with unit prices and stock status. Server parses these responses, extracts price and inventory fields, and updates internal records to include cost and availability attributes for each candidate purchase item.

### Step 11:

Server refines the meal plan based on budget and purchasing constraints using the generative AI model.

Input: Structured data including existing items, time information, price data, inventory data, and budget information.

Output: Updated meal plan information with candidate purchase items conforming to the budget.

Server generates a second prompt sentence that summarizes available ingredients, desired cooking times, available shopping window, prices, inventory, and user budget. Server again tokenizes this prompt and inputs it into the generative AI model. Model inference produces a refined textual plan specifying which additional items should be purchased within the budget and how they are used in menus. Server parses this text to identify proposed purchase items and associated menus, calculates total cost using stored price information, and removes or adjusts proposed items if necessary to ensure that total cost does not exceed the budget.

### Step 12:

Server generates and transmits purchase processing information to the electronic commerce platform.

Input: Selected candidate purchase items and associated purchase parameters.

Output: Purchase processing request sent to the electronic commerce platform.

Server compiles a purchase list that includes item identifiers, required quantities, total estimated cost, and user delivery address. Server converts this list into a transaction request message according to the commerce platform's protocol and transmits it via the communication network. Based on the platform response, server records order identifiers and status in the database. This step transforms structured item selections into a concrete electronic order.

### Step 13:

Server determines desired delivery times and coordinates delivery with a delivery management platform.

Input: Meal plan execution times and purchase order information.

Output: Delivery request information submitted to a delivery management platform.

Server compares the execution times of menus requiring purchased items with estimated delivery windows available from the commerce or delivery systems. Server computes a desired delivery time that ensures purchased items arrive before their first planned use, possibly applying a margin for preparation or delays. Server constructs delivery request data including item list, destination, and desired delivery time, and sends this data to a delivery management platform using its interface. Server stores the resulting delivery confirmation or tracking information.

### Step 14:

Server sends meal plan display data to the terminal.

Input: Structured meal plan information including menus, schedules, and purchase/delivery status.

Output: Display-ready data received by the terminal.

Server formats the meal plan information into a structure optimized for user viewing, such as grouping menus by date and indicating fulfillment status (for example, available now, awaiting delivery). Server may add simplified text descriptions and sequence steps for each recipe. Server encapsulates this formatted data in a response message and transmits it to the terminal over the communication network.

### Step 15:

Terminal displays the meal plan and interacts with the user for further refinement.

Input: Display-ready meal plan data from the server.

Output: User actions and potential refinement instructions.

Terminal parses the received data and renders the menus on its display, for example as a calendar view or list view. User reviews the proposed menus and may select options for more details, such as viewing ingredients and steps. User may also specify changes, such as excluding a particular ingredient or requesting additional vegetarian options. Terminal converts these user refinements into structured constraint data and sends them back to the server, where the server can update structured data, reconstruct a new prompt sentence, and repeat the generative process to produce a revised meal plan.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

Conventional computer-implemented recipe and shopping assistants typically handle refrigerator contents, user constraints, and recipe generation in a fragmented and ad-hoc manner. In many cases, a computing device merely receives manual text input of ingredients and user preferences, and then performs a simple database lookup or invokes a content generation service. Such approaches rely heavily on user effort, are error-prone due to inaccurate or incomplete ingredient entry, and do not fully exploit automated recognition and generation capabilities. As a result, the computing resources are not efficiently used, and the user experience degrades when the system fails to generate coherent meal plans aligned with actual available items and practical shopping constraints.

Furthermore, in known systems that incorporate machine learning or content generation, the interaction with a generative artificial intelligence model is often limited to unstructured prompts manually composed by the user or by simple templates. The prompt generation logic does not systematically transform heterogeneous data sources such as image-based object recognition results and structured user condition data into a context-rich, machine-targeted prompt sentence. Consequently, the generative model may produce suboptimal or irrelevant meal plans, leading to repeated calls, unnecessary network traffic, and increased processing overhead. This results in inefficient utilization of both local processing resources and remote model resources.

Additionally, conventional systems do not tightly integrate the downstream processing steps of identifying missing items, mapping them to purchasable merchandise, and orchestrating delivery through external services as part of a single, coherent computational workflow. Instead, such steps are often performed manually by the user or through disconnected applications. This fragmentation forces the user to re-enter similar information multiple times, increases latency between planning and ordering, and introduces inconsistencies between the generated recipes and the actually procured items. From a computer-technology standpoint, this leads to duplicated processing, redundant external calls, and complex error handling across multiple independent systems.

In summary, there is a need for an improved computer-implemented technique that (i) automatically acquires and processes image data of stored objects, (ii) structures user constraints as machine-processable condition information, (iii) algorithmically constructs a prompt sentence tailored for a generative artificial intelligence model by integrating these data, and (iv) systematically performs comparison, purchase, and delivery orchestration based on the generated meal plan. Such a technique should reduce manual input, decrease computational redundancy, improve the relevance and consistency of generated content, and enhance overall efficiency in the use of processing and network resources.

The specific processing by the specific processing unit 290 of the data processing device 12 in Application Example 1 is realized by the following means.

The present invention provides a server comprising a processor configured to acquire image data of a storage apparatus from an image acquisition device, preprocess the image data by using an image analysis algorithm to convert the image data into numerical data, identify stored objects on a basis of the numerical data to generate stored object information, cause a terminal device to display a user interface, receive user input including time information required for cooking, shopping availability period information, meal provision period information, and meal provision frequency information, convert the user input into condition information as structured data and store the condition information, acquire the stored object information and the condition information, integrate the stored object information and the condition information into natural language text, generate a prompt sentence for input to a generative artificial intelligence model, transmit the prompt sentence to the generative artificial intelligence model via a network, receive response text as meal plan information from the generative artificial intelligence model, analyze the meal plan information to extract dish name information, required stored object information, and cooking procedure information, compare the required stored object information with the stored object information to specify shortage stored object information, convert the meal plan information, the stored object information, and the shortage stored object information into display data capable of being output to the terminal device, associate the shortage stored object information with merchandise information, acquire price information and inventory information from an external information providing apparatus on a basis of the merchandise information, automatically generate purchase candidate information on a basis of the price information and budget information, execute purchase processing via an external transaction processing apparatus on a basis of the purchase candidate information, generate delivery request information on a basis of the shortage stored object information and delivery request time information specified by the user, transmit the delivery request information to an external delivery management apparatus to perform delivery processing, acquire delivery result information from the external delivery management apparatus, and output the delivery result information to the terminal device. This enables the server to implement an integrated computational workflow that automatically transforms raw image data and structured user constraints into an optimized prompt sentence for a generative artificial intelligence model, obtains a consistent meal plan in response, and efficiently coordinates subsequent purchase and delivery processing, thereby reducing manual operations, lowering redundant processing and network traffic, and improving overall efficiency and reliability of computer-implemented meal planning and procurement.

The term “processor” refers to a hardware computing element or a combination of hardware computing elements, such as a central processing unit, a graphics processing unit, a digital signal processor, or a plurality thereof, that executes instructions to perform data processing operations described in this specification.

The term “system” refers to an arrangement comprising at least the processor and one or more associated components, such as memory, storage, communication interfaces, and peripheral devices, configured to cooperatively execute the functions described in this specification.

The term “storage apparatus” refers to a physical container or environment in which items are stored under controlled or uncontrolled conditions, such as a refrigerator, cabinet, pantry, or other storage facility for foodstuffs or consumables.

The term “image acquisition device” refers to a device capable of capturing image data representing a scene, such as a camera of a mobile terminal, a dedicated imaging sensor, or any imaging unit configured to output digital image data.

The term “image data” refers to digital data representing a still image or a sequence of images, including pixel values encoded in one or more formats, such as bitmap, raster, or compressed image formats.

The term “image analysis algorithm” refers to a software-implemented procedure or model that processes image data to extract information, including but not limited to object detection, object classification, segmentation, or feature extraction.

The term “numerical data” refers to data represented in a numerical format, such as arrays, tensors, vectors, or matrices of numerical values, that are suitable for computational processing by the processor or by a machine learning model.

The term “stored objects” refers to physical items contained within the storage apparatus, such as food items, ingredients, or other articles, that can be recognized by processing of image data.

The term “stored object information” refers to data describing one or more stored objects identified by processing of the image data, including but not limited to labels, categories, quantities, or confidence levels.

The term “terminal device” refers to an electronic device operated by a user, such as a smartphone, tablet, personal computer, or other user-facing device capable of presenting a user interface and communicating with the server.

The term “user interface” refers to a graphical, textual, voice-based, or other interaction mechanism presented on the terminal device that allows the user to input data or receive output.

The term “time information required for cooking” refers to data indicating a time constraint or duration within which the user wishes to prepare or complete a cooking activity.

The term “shopping availability period information” refers to data indicating one or more time periods during which the user is able or willing to perform shopping or receive items.

The term “meal provision period information” refers to data indicating a date range or time range over which meals are to be planned or provided.

The term “meal provision frequency information” refers to data indicating a count, schedule, or frequency of meals to be prepared or consumed within a specified period.

The term “condition information” refers to structured data derived from user input, including time constraints, shopping availability, meal period, meal frequency, budget, or other requirements to be used in planning meals.

The term “structured data” refers to data organized according to a defined format, schema, or data model, such as key-value pairs, records, or objects, that can be systematically processed by a computer program.

The term “natural language text” refers to text expressed in a human language, such as English or another spoken language, suitable for interpretation by a human reader or a language-processing model.

The term “prompt sentence” refers to natural language text constructed to be supplied as input to a generative artificial intelligence model, the text specifying context, constraints, or instructions that guide generation of an output.

The term “generative artificial intelligence model” refers to a computation model, such as a machine learning model or neural network, configured to generate text or other content in response to input, including but not limited to large language models.

The term “meal plan information” refers to information generated by the generative artificial intelligence model or by subsequent processing, including dish names, required ingredients, schedules, and cooking steps.

The term “dish name information” refers to data identifying one or more proposed dishes, meals, or recipes, typically represented as natural language names or titles.

The term “required stored object information” refers to data describing one or more items or ingredients required to prepare a dish or meal, including their identities and optionally their quantities or forms.

The term “cooking procedure information” refers to data describing one or more steps, instructions, or methods for preparing, cooking, or assembling a dish or meal.

The term “shortage stored object information” refers to data indicating one or more items or ingredients that are required for a dish or meal and are not present, or not present in sufficient quantity, among the stored objects identified in the storage apparatus.

The term “display data” refers to data formatted for visual or other presentation on the terminal device, including text, icons, lists, or other user interface elements.

The term “merchandise information” refers to data representing purchasable items offered by a supplier or service, including product identifiers, names, categories, and packaging units corresponding to stored objects or ingredients.

The term “external information providing apparatus” refers to a computing system or service accessible via a network, configured to provide information such as prices, inventory levels, or product details associated with merchandise.

The term “price information” refers to data representing monetary values associated with merchandise, including unit prices, total prices, and discounts.

The term “inventory information” refers to data indicating availability status, stock quantities, or other supply-related information for merchandise.

The term “budget information” refers to data indicating a financial limit or range specified by the user or by a system policy for purchasing items.

The term “purchase candidate information” refers to data representing one or more recommended items or combinations of items selected for possible purchase, based on shortage stored object information, price information, budget information, or other constraints.

The term “external transaction processing apparatus” refers to a computing system or service, accessible via a network, that executes financial or commercial transactions, such as order placement, payment processing, and order confirmation.

The term “delivery request information” refers to data specifying items to be delivered, quantities, destination information, and timing constraints for a delivery operation.

The term “delivery request time information” refers to data indicating a desired delivery date, time, or time window specified by the user or derived from user preferences.

The term “external delivery management apparatus” refers to a computing system or service, accessible via a network, configured to manage logistics operations, including acceptance, scheduling, routing, and tracking of deliveries.

The term “delivery result information” refers to data indicating one or more outcomes of a delivery request, including acceptance, scheduled time, tracking identifiers, completion status, or failure information.

In one embodiment, a server cooperates with a terminal and a user to implement the claimed system. The server includes at least one processor, a main memory, a non-volatile storage device, and a network interface. The terminal includes a processor, a display, a user input device such as a touch panel, and an image acquisition device such as an integrated digital camera. The user operates the terminal in a physical environment in which a storage apparatus, for example a refrigerator, stores physical items such as food ingredients.

The server uses a software stack including an operating system, a web application framework, a database management system, and a machine-learning runtime. As one example, the server runs a Linux operating system, a web framework such as a general-purpose HTTP server, a relational database system, and a machine-learning framework such as an implementation of a tensor-based numerical computation library. The terminal uses an operating system such as a smartphone operating system, an application runtime, and a camera framework.

The server executes a program that implements multiple cooperating modules: an image ingestion module, an image analysis module, a stored-object management module, a condition management module, a prompt generation module, a generative AI interaction module, a meal-plan analysis module, a shortage-item determination module, a merchandise mapping module, a purchase orchestration module, and a delivery orchestration module. Each module uses specific data structures and algorithms, as described below.

The terminal captures image data of the storage apparatus when the user points the camera at the interior of the storage apparatus and activates a capture control. The terminal encodes the captured image into a digital image file, such as a JPEG file, and transmits the file to the server via a network. The server receives the file and stores it in non-volatile storage, associating it with a user identifier. In this way, the system directly couples to a real-world storage apparatus and acquires a faithful digital representation of its contents; as a result, subsequent computations operate on physically grounded data rather than on manually entered approximations.

The server uses the image analysis module to convert the image data into numerical data and to identify stored objects. In one embodiment, the server uses a convolutional neural network implemented by a tensor computation framework. The server represents each image as a floating-point tensor of shape [1, H, W, 3], where H and W are normalized spatial dimensions, for example 224 by 224. The server performs normalization operations including channel-wise mean subtraction and variance scaling, thereby converting raw pixel intensities into features that are more suitable for convolution operations. The server applies a sequence of convolutional layers, each followed by non-linear activation functions, for example rectified linear units, and pooling layers, to compute feature maps. The server then applies one or more fully connected layers and a final softmax layer over a vocabulary of object classes corresponding to generic ingredient categories such as “leafy vegetable,” “dairy product,” or “fruit.” Optionally, the server uses a region proposal network to localize multiple objects within one image. The server thus obtains numerical class probability distributions and bounding box coordinates.

The server converts the numerical output to stored object information by mapping class identifiers to abstract categories and, optionally, to canonical ingredient descriptors. The server prunes low-confidence detections by applying a threshold to the probability values and uses non-maximum suppression to avoid duplicate detections of the same physical object region. As a result, the server constructs a structured list of stored objects, for example a list of records each containing a category label, a confidence score, and a spatial region. The server stores this list in the database as structured data. Because the server uses a trained neural network and statistical thresholds to determine which objects are recognized, the server reduces human mis-entry of ingredient lists and improves recognition accuracy compared to simple template-based or keyword-based recognition.

The server trains the convolutional neural network in advance using a supervised learning procedure. The server obtains a training dataset of labeled images of storage apparatus interiors. The server performs data augmentation operations such as random cropping, rotation, color jittering, and horizontal flipping to increase generalization and reduce overfitting. During training, the server computes a cross-entropy loss between predicted class distributions and ground-truth class labels and uses stochastic gradient descent or an adaptive gradient-based optimizer to update network weights. The server applies backpropagation to propagate the loss gradient through the network layers. The server may store learned weight parameters in a model file on disk. By training the model in this way, the server achieves higher recognition precision and recall when applied to user images, leading to more accurate downstream meal planning and fewer unnecessary purchase recommendations.

The terminal provides a user interface that allows the user to specify condition information.

The terminal displays a set of controls such as time pickers, calendars, sliders, and text fields.

The user can enter time information required for cooking, shopping availability period information, meal provision period information, and meal provision frequency information.

The terminal converts these inputs into structured data, for example a key-value representation, and transmits them to the server via the network. The server receives this condition information, validates the data types and ranges, and stores the information in the database associated with the user.

The server uses a prompt generation module to combine the stored object information and the condition information into a prompt sentence for a generative AI model. The server applies a rule-based natural language construction algorithm. In one embodiment, the server uses a template system with slots for ingredient categories, quantities if available, time constraints, and shopping constraints. The server may perform abstraction of specific object names to higher-level ingredient categories to reduce unnecessary variation and to improve the stability of generative model outputs. The server also may specify output formatting requirements in the prompt sentence, such as requesting a structured enumeration of ingredients and cooking steps.

For example, the server may generate a prompt sentence such as:

“The user has the following ingredients in the refrigerator: tomato, cheese, and basil. The user wants one dinner recipe that can be prepared within 30 minutes. The user can buy additional ingredients this evening between 18:00 and 20:00. Propose one optimal recipe, and clearly list: (1) the recipe name, (2) all required ingredients and quantities, (3) which ingredients are already available, (4) which ingredients must be purchased, and (5) step-by-step cooking instructions.”

In another example, the server may generate a prompt sentence such as:

“There are tomatoes, cheese, and basil in the refrigerator. Please propose a dish that can be prepared within 30 minutes, using mainly these ingredients. Include a short recipe, a complete ingredient list, and simple cooking steps.”

The server sends the prompt sentence as part of a structured request to a generative AI model hosted by an external computation service. The generative AI model is, for example, a large language model implemented as a multi-layer transformer network. The transformer network uses an encoder-decoder or decoder-only architecture with self-attention mechanisms. The model processes tokenized text sequences and predicts subsequent tokens based on learned probability distributions. The model is pre-trained on large corpora of text and optionally fine-tuned on domain-specific recipe data.

During operation, the server does not treat the generative AI model as a black box; instead, the server constrains the model's operation through the prompt structure and through additional decoding parameters such as maximum output length, temperature, and nucleus sampling threshold. The server may also specify special delimiter tokens in the prompt sentence to encourage the model to separate sections of the output, such as “Recipe Name:”, “Ingredients:”, and “Steps:”. By carefully structuring both the prompt sentence and the decoding parameters, the server improves the consistency, parse-ability, and technical utility of the generated text. This reduces the need for complex error handling and repeated queries, thereby decreasing network traffic and processing overhead.

The server receives the generated response text from the generative AI model and analyzes it using a meal-plan analysis module. The server parses the response to extract dish names, required ingredients, and cooking steps. In one embodiment, the server uses a finite-state parser that looks for predefined markers and patterns such as line prefixes or bullet points. In another embodiment, the server uses a secondary, smaller language model or a rule-based parser configured to convert the free-form response into a normalized, structured representation. The server then stores this representation as meal plan information in the database. Because the server uses a deterministic parsing scheme aligned with the prompt design, the system can reliably transform unstructured natural language output into machine-readable records. This provides a technical improvement over naive text generation systems that leave post-processing to ad-hoc string matching.

The server uses the shortage-item determination module to compare the required stored object information with the stored object information initially obtained from the storage apparatus. The server represents both sets as normalized ingredient categories and applies set difference operations. If needed, the server uses a mapping table or ontology that relates synonyms and variant expressions to canonical categories, thus preventing artificial mismatches due to vocabulary differences between the generative AI model and the image analysis module. The server outputs shortage stored object information indicating which ingredients are missing or insufficient. The server stores these results in the database and forwards them to subsequent modules.

The server maps the shortage stored object information to merchandise information provided by external services. The server sends search queries to an external information providing apparatus over a network, specifying canonical category names and optional constraints such as organic, size, or brand preferences. The external apparatus responds with product identifiers, names, unit sizes, prices, and inventory data. The server selects appropriate items, for example by applying a scoring function that balances price and quantity, and generates purchase candidate information that minimizes unnecessary over-ordering while ensuring that required ingredients are acquired. By using canonical categories and a scoring algorithm, the server reduces errors in item selection and improves the efficiency of the overall purchasing process.

The server can further generate delivery request information by combining purchase candidate information with delivery request time information specified by the user. The server communicates with an external delivery management apparatus via a network interface, sending structured delivery requests that include destination address, preferred time windows, and item lists. The external apparatus returns delivery result information such as accepted time windows, tracking identifiers, or exceptions. The server stores and then transmits such information to the terminal. The terminal displays this information so that the user can see both the planned meal and the associated deliveries in a unified interface.

The terminal receives meal plan information, stored object information, shortage stored object information, purchase candidate information, and delivery result information from the server. The terminal renders these as visual elements on the display, such as lists of dishes, lists of ingredients the user already has, and lists of items that will be delivered. The user can review the proposals and optionally adjust preferences through the user interface. Because the server has already normalized and integrated the data, the terminal's rendering logic can remain comparatively simple, reducing processing demands on the terminal and minimizing bandwidth consumption through compact, structured responses.

From a technical perspective, this configuration provides several improvements to computer technology. First, the server replaces manual text-based entry of ingredients with automated image analysis using a specifically trained neural network. This reduces the frequency of user input errors and accelerates the acquisition of structured data. Second, the server uses a non-conventional combination of image-based stored object information, time-based condition information, and rule-based prompt construction to generate a highly constrained and context-rich prompt sentence for the generative AI model. This combination enables the generative AI model to produce outputs that are easier to parse and more directly actionable, reducing the number of round-trip communications and the computational cost of repeated queries.

Third, the server uses explicit data structures for stored objects, condition information, and meal plan information and processes them through a defined pipeline. Each stage of the pipeline performs a well-specified transformation, such as tensor normalization, softmax-based classification, ontology-based canonicalization, template-based text generation, and deterministic parsing. This architectural design reduces ambiguity, simplifies error recovery, and increases the reproducibility of system behavior, which are technical benefits beyond simple automation of human decision-making.

Fourth, the server integrates purchase and delivery orchestration into the same computational pipeline that generates meal plans. By computing shortage stored object information directly from recognized and generated data, the server avoids repeated manual re-entry of shopping lists. The server uses optimized network access patterns to external services, for example sending batched requests or caching product mappings, which can reduce network latency and bandwidth consumption. As a result, the total time and resource usage required to translate ingredient recognition into confirmed deliveries is reduced.

The server can further apply variants of the described embodiment. In one variant, the server uses a different network architecture for image analysis, such as a residual network or a vision transformer, to improve recognition performance under particular lighting or occlusion conditions. In another variant, the server refines the generative AI model prompts by incorporating feedback data about previous user selections, thus dynamically adjusting prompt content to favor recipes that are more likely to be accepted. In still another variant, the server executes part of the image analysis or meal-plan analysis on an edge device or gateway located physically closer to the storage apparatus, such as a smart appliance controller, thereby reducing latency and distributing computational load.

In each embodiment, the server, the terminal, and the user cooperate through specific technical mechanisms: acquisition of image data from a physical storage apparatus, transformation of that data into machine-interpretable numerical representations, context-sensitive prompt sentence construction for a generative AI model, structured parsing of generated content, and automated mapping of missing items to real-world merchandise and delivery processes. Because these mechanisms rely on carefully designed neural network architectures, training procedures, data structures, and transformation rules, the system improves the speed, accuracy, and resource efficiency of computer-implemented meal planning and procurement, and is not limited to a mere automation of human cognitive steps.

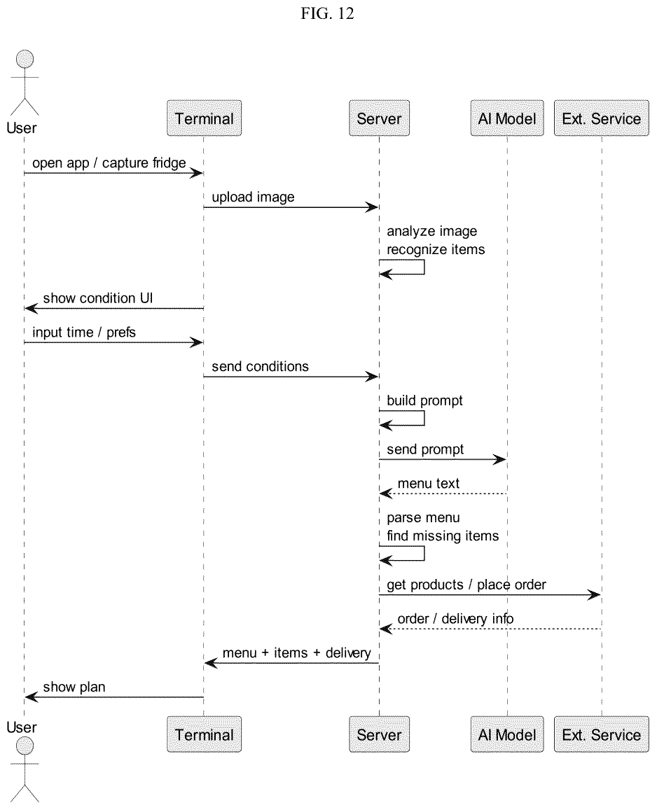

The following describes the processing flow using FIG. 12.

### Step 1:

The user operates the terminal to launch an application and activate a camera function. The terminal displays a live preview from an image acquisition device and receives a capture command from the user. The input is a real-world scene inside a storage apparatus, and the output is raw image data encoded as a digital image file. The terminal converts the optical signal captured by the imaging sensor into pixel data, encodes the pixel data into a compressed image format, and stores the resulting file in a temporary storage area.

### Step 2:

The terminal transmits the captured image file to the server via a network connection. The input is the compressed image file and user identification information, and the output is an HTTP request containing the image as a payload. The terminal opens a network socket, constructs a request with headers including content type and authentication data, and writes the image bytes into the request body for delivery to the server.

### Step 3:

The server receives the HTTP request and extracts the image file. The input is the HTTP request containing the image data, and the output is a stored image record associated with a user identifier. The server parses the request, validates the content type, writes the image bytes to non-volatile storage, and inserts a database record that links the storage path of the image to the user.

### Step 4:

The server preprocesses the stored image and converts it into numerical data suitable for analysis. The input is the stored image file, and the output is a normalized tensor representing pixel intensities. The server decodes the compressed image into a two-dimensional pixel array, resizes the array to a fixed width and height, converts integer pixel values into floating-point values, and applies normalization operations such as mean subtraction and scaling so that the values fall within a predetermined numeric range.

### Step 5:

The server identifies stored objects by applying a trained neural network to the normalized tensor. The input is the normalized tensor, and the output is stored object information describing categories present in the image. The server feeds the tensor into a convolutional neural network, performs a sequence of convolution, activation, and pooling operations to compute feature maps, applies one or more fully connected layers and a softmax function to produce class probability distributions, and applies thresholds and non-maximum suppression to select categories and associated confidence values representing stored objects.

### Step 6:

The server records the identified stored objects as structured data. The input is the set of detected categories and associated metadata, and the output is a stored object list in a database. The server maps class identifiers to generic ingredient categories, constructs data records including category labels, confidence scores, and optional bounding box coordinates, and inserts these records into a database table keyed by the user and by the original image.

### Step 7:

The terminal presents a user interface for condition input and receives user preferences. The input is user interaction events with the interface components, and the output is structured condition information ready for transmission. The terminal displays controls for cooking time limits, shopping availability periods, meal provision periods, and meal frequencies, converts the user's selections and entries into a key-value structure, validates that values fall within allowable ranges, and prepares the resulting structure for network transfer.

### Step 8:

The terminal transmits the structured condition information to the server. The input is the validated key-value structure representing user conditions, and the output is an HTTP request containing a serialized representation of these conditions. The terminal encodes the structure as a text-based format, attaches it to a request body, and sends the request to a designated endpoint exposed by the server.

### Step 9:

The server receives and stores the condition information. The input is the HTTP request containing the serialized condition data, and the output is a normalized condition record in the database. The server parses the request payload, checks field types and mandatory fields, normalizes formats for dates and times, and inserts or updates a database record that associates the condition information with the user.

### Step 10:

The server retrieves the stored object information and the condition information and prepares them for prompt construction. The input is the stored object list and the normalized condition record, and the output is an internal representation that combines both datasets. The server loads the relevant records from the database, converts them into a unified in-memory structure, and resolves any conflicts such as duplicate or obsolete entries.

### Step 11:

The server generates a prompt sentence for a generative AI model by applying a rule-based text construction algorithm. The input is the unified in-memory representation of stored objects and conditions, and the output is a natural language prompt sentence. The server selects a template, fills slots with ingredient categories and constraint values, inserts connecting phrases, and optionally adds section markers to instruct the model on how to structure its response. For example, the server may generate the prompt sentence: “The user has the following ingredients in the refrigerator: tomato, cheese, and basil. The user wants one dinner recipe that can be prepared within 30 minutes. The user can buy additional ingredients this evening between 18:00 and 20:00. Propose one optimal recipe, and clearly list: (1) the recipe name, (2) all required ingredients and quantities, (3) which ingredients are already available, (4) which ingredients must be purchased, and (5) step-by-step cooking instructions.”

### Step 12:

The server sends the prompt sentence to a generative AI model via a network interface. The input is the prompt sentence and configuration parameters for the generative AI model, and the output is a request message delivered to a model execution service. The server tokenizes the text if necessary, wraps the tokens and configuration parameters into a structured request object, establishes a secure connection to the external model service, and transmits the request over the network.

### Step 13:

The server receives generated meal plan information from the generative AI model. The input is a response message containing generated text, and the output is a raw meal plan description expressed in natural language. The server reads the response, extracts the generated content field, decodes any encoded characters, and reconstructs the full text of the meal plan as returned by the model.

### Step 14:

The server parses the generated meal plan text into structured components. The input is the natural language meal plan description, and the output is structured meal plan information comprising dish names, required ingredients, and cooking steps. The server scans the text for markers such as headings and bullet points, separates sections that describe the recipe name, the ingredient list, and the procedure, and builds a structured representation in which each section is stored as a distinct field or list.

### Step 15:

The server determines shortage stored objects by comparing required ingredients with recognized stored objects. The input is the structured ingredient list from the meal plan and the stored object list obtained from image analysis, and the output is shortage stored object information. The server normalizes ingredient names to canonical categories, constructs sets of available and required categories, and computes the set difference to identify ingredients that are absent or insufficient in the storage apparatus.

### Step 16:

The server records the shortage stored object information and prepares it for further processing. The input is the computed set of missing ingredient categories, and the output is a structured shortage record stored in the database. The server associates each missing category with the corresponding recipe identifier, attaches quantity estimates if provided by the meal plan, and inserts the resulting records into a dedicated table.

### Step 17:

The server maps the shortage stored object information to merchandise information obtained from an external service. The input is the shortage record and, optionally, user preferences or constraints, and the output is a list of candidate merchandise items. The server queries an external information providing apparatus with category names or search keywords, receives product data including identifiers, unit sizes, prices, and availability, and selects one or more items for each missing category based on predefined selection rules or scoring criteria.

### Step 18:

The server generates purchase candidate information and, when appropriate, executes purchase processing. The input is the list of candidate merchandise items and budget information associated with the user, and the output is a set of confirmed items and a purchase transaction request. The server compares item prices against budget limits, filters out items that would cause the total cost to exceed a threshold, and composes a final shopping list that satisfies the shortage while remaining within the budget. The server forwards this list to an external transaction processing apparatus as an order request.

### Step 19:

The server orchestrates delivery by generating delivery request information based on the confirmed purchase items and the user's delivery preferences. The input is the confirmed shopping list and delivery request time information, and the output is a delivery request sent to an external delivery management apparatus. The server compiles destination details, desired delivery windows, and item identifiers into a structured delivery request, sends it over the network, and receives delivery result information such as confirmed time windows and tracking identifiers.

### Step 20:

The server aggregates meal plan information, stored object information, shortage stored object information, and delivery result information and prepares display data for the terminal.

The input is the set of structured records stored in the database for a given user session, and the output is a compact representation suitable for rendering on the terminal. The server formats the data into lists and sections, reduces redundant fields, and serializes the result into a structure that indicates which ingredients the user already has, which items are being purchased and delivered, and how to prepare the proposed dish.

### Step 21:

The terminal receives the display data from the server and renders it on the display for the user. The input is the structured display data including meal plans and logistics information, and the output is a user interface showing recommended dishes, ingredient availability, and delivery status. The terminal parses the data, creates visual elements such as headings, lists, and buttons, and updates the screen so that the user can view and interact with the proposed meal and procurement plan.

### Step 22:

The user reviews the presented information on the terminal and may perform follow-up actions such as confirming or rejecting recommendations. The input is the visual representation of the meal plan and procurement details, and the output is user commands that may adjust or finalize the plan. The user may select confirmation controls, modify constraints, or request an alternative proposal, and the terminal captures these commands and transmits them back to the server, allowing the system to refine or complete the computational workflow.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

Conventional meal planning and grocery assistance systems primarily focus on presenting static recipe lists or simple recommendation results based on user-selected options. In such systems, the computing apparatus typically performs only straightforward retrieval or filtering of pre-stored recipe data and does not deeply integrate real-time perception of available ingredients, flexible user constraints, and automated transaction handling in a unified technical workflow. As a result, these systems do not fully exploit advanced machine learning models, and the overall processing remains fragmented and inefficient from a computer-technology standpoint.

In particular, known systems exhibit the following technical problems. First, the acquisition and utilization of image information from a storage apparatus, such as a refrigerator, are often treated as stand-alone functions, and the extracted ingredient information is not consistently converted into normalized structured data suitable for subsequent automated reasoning and generation processes. This leads to brittle pipelines in which downstream components cannot reliably consume the ingredient information, thereby requiring manual user intervention and limiting machine-to-machine interoperability.

Second, existing systems do not define a systematic mechanism by which a processor transforms heterogeneous input information such as image information, ingredient information, and diverse meal condition information including time constraints, nutritional conditions, and budget constraints into a well-formed prompt sentence optimized for a generative AI model. Without such structured prompt generation, the interaction with a generative AI model is ad hoc, non-repeatable, and heavily dependent on manual crafting of prompts, which reduces reliability, reproducibility, and scalability of the computer system.

Third, conventional architectures do not provide an integrated technical framework to automatically interpret the output of a generative AI model into structured meal plan information, compare the required ingredient information against available ingredient information, and algorithmically derive shortage ingredient information. As a consequence, the system cannot automatically prepare purchase-related data in a machine-usable format, and the linkage between content generation and transaction processing remains weak and error-prone.

Fourth, from a transaction and logistics perspective, existing systems do not sufficiently formalize the processor's role in coordinating with external commerce systems. In many cases, a user must manually transfer recipe requirements into a separate ordering interface.

This manual translation step introduces errors, interrupts the digital workflow, and prevents the computing system from performing end-to-end automation of order generation, order confirmation handling, and delivery scheduling based on precise meal condition information, such as desired meal execution time and cooking time constraints.

Fifth, conventional systems do not provide a technical mechanism to iteratively refine interaction with a generative AI model based on user feedback, user preference history information, and updated ingredient information. Without a processor-controlled feedback loop that regenerates prompt sentences and re-queries the generative AI model, the system cannot adapt over time, and it cannot efficiently converge to meal plans that better match the user's evolving preferences and constraints. This also means that the system is unable to leverage accumulated usage history as a resource for improving subsequent computational outputs.

In summary, there is a need for an improved computer-implemented system and method that: (i) acquires and normalizes ingredient information from image information of a storage apparatus, (ii) algorithmically generates structured prompt sentences for a generative AI model based on unified ingredient and condition information, (iii) parses and structures the generative AI model responses into machine-usable meal plan information and shortage ingredient information, and (iv) tightly integrates this information with automated ordering and delivery coordination through external commerce and logistics systems. Such an improved system should provide a technical effect of reducing manual operations, enhancing data consistency throughout the processing pipeline, improving the effective utilization of generative AI models, and enabling a more efficient and reliable end-to-end computation flow on the server side.

The specific processing by the specific processing unit 290 of the data processing device 12 in Example 2 is realized by the following means.

The present invention provides a server comprising a processor and a memory storing instructions that, when executed by the processor, cause the processor to acquire image information indicating a state inside a storage apparatus via an image acquisition device or a portable information terminal; to execute image analysis processing on the image information using an image recognition program so as to identify ingredient information present inside the storage apparatus and convert the ingredient information into structured data as standardized ingredient information; to acquire, based on the image information and an operation of a user received from the portable information terminal, meal condition information including at least one of a meal execution time, a cooking required time, a number of persons, a nutritional condition, a preference condition, a budget condition, and a cooking time constraint; to generate, based on the standardized ingredient information and the meal condition information, a prompt sentence for input to a generative AI model by using template information and a character string generation algorithm; to transmit the prompt sentence via a communication network to a generative information processing model implemented on an external apparatus or within the server, to cause the generative AI model to generate meal plan information based on the prompt sentence, and to receive response information including the meal plan information; to parse the meal plan information so as to extract dish name information, step information, and required ingredient information, and to compare the required ingredient information with the standardized ingredient information so as to calculate shortage ingredient information; to structure display information including at least the meal plan information and the shortage ingredient information; and to transmit the display information to the portable information terminal and thereby cause the portable information terminal to present, via a user interface, the meal plan information and the shortage ingredient information such that the user can browse, edit, and confirm order candidate information, and to acquire the order candidate information edited or confirmed by the user and store the order candidate information in association with the shortage ingredient information. This enables the server to implement an end-to-end, computer-controlled workflow that transforms raw image information into normalized ingredient data, automatically generates optimized prompt sentences for the generative AI model, converts the resulting meal plan information into structured representations linked to shortage ingredient information, and tightly integrates these representations with a user-editable ordering interface, thereby improving the technical efficiency, reliability, and automation level of meal planning and ingredient procurement processing executed by the computer system.

The term “processor” refers to a hardware information processing unit, such as a central processing unit or a programmable logic device, configured to execute instructions to perform arithmetic, logical, and control operations described in the present specification.

The term “memory” refers to a hardware storage medium, such as a semiconductor memory, magnetic storage device, or optical storage device, capable of storing instructions and data for access by the processor.

The term “storage apparatus” refers to an apparatus for storing food items or ingredients under controlled environmental conditions, such as a refrigeration device, freezing device, or similar food storage equipment.

The term “image acquisition device” refers to a hardware device capable of capturing image information, such as a camera module, an image sensor, or a similar imaging component.

The term “portable information terminal” refers to a mobile electronic device, such as a smartphone, tablet, or handheld computing device, that includes a display, an input interface, and a communication function for interacting with a server.

The term “image information” refers to digital data representing a visual scene, such as still image data or video frame data, that indicates a state inside the storage apparatus.

The term “image recognition program” refers to software configured to perform image analysis processing, including object detection, object classification, or label extraction, on image information to identify objects such as ingredients.

The term “ingredient information” refers to information indicating the presence, type, or quantity of food items or components detected within the storage apparatus.

The term “standardized ingredient information” refers to ingredient information that has been normalized to canonical names or categories according to predefined rules or a reference database, and represented in a structured format suitable for further processing.

The term “structured data” refers to data organized according to a defined schema, such as key-value pairs, lists, or records, which enables programmatic access and processing by the processor.

The term “meal condition information” refers to constraint information, requirement information, or preference information related to preparation and consumption of a meal, including at least one of a meal execution time, a cooking required time, a number of persons, a nutritional condition, a preference condition, a budget condition, and a cooking time constraint.

The term “meal execution time” refers to a point in time or a time range by which a meal is intended to be ready for consumption.

The term “cooking required time” refers to an estimated duration needed to perform preparation and cooking steps to complete a meal.

The term “number of persons” refers to information indicating a count of people for whom the meal is to be prepared.

The term “nutritional condition” refers to constraints or targets relating to nutritional properties of a meal, such as caloric intake, macronutrient balance, or inclusion or exclusion of specific nutrients.

The term “preference condition” refers to conditions reflecting user preferences, such as taste preferences, cuisine types, dietary restrictions, or ingredient preferences.

The term “budget condition” refers to cost-related constraints, such as a maximum allowable expenditure for purchasing ingredients for a meal.

The term “cooking time constraint” refers to a limitation on total allowable preparation and cooking time for a meal, which may be derived from or combined with the meal execution time and the cooking required time.

The term “template information” refers to predefined text patterns, formats, or structures that the processor uses as a basis for generating a prompt sentence by inserting or combining variable elements such as ingredient information and meal condition information.

The term “character string generation algorithm” refers to a software procedure or rule set executed by the processor to construct, arrange, and output text strings, including the generation of a prompt sentence from template information and input parameters.

The term “prompt sentence” refers to a text string or sequence of text strings formatted for input to a generative AI model, the content of which encodes ingredient information, meal condition information, and instructions for generating meal plan information.

The term “generative AI model” refers to a machine-learned information processing model configured to generate output information, such as natural language text, based on input information including a prompt sentence.

The term “generative information processing model” refers to a generative AI model or similar algorithmic model that produces new data or content, such as meal plan information, from provided input information.

The term “meal plan information” refers to information output from the generative AI model that describes one or more meal proposals, including dish information, required ingredients, and cooking procedures.

The term “response information” refers to data received from the generative AI model or a generative information processing model providing apparatus in response to a transmitted prompt sentence, the data including at least meal plan information.

The term “dish name information” refers to text information representing the names or titles of dishes included in the meal plan information.

The term “step information” refers to procedural information describing ordered cooking steps, actions, or instructions for preparing a dish.

The term “required ingredient information” refers to information indicating ingredients needed to prepare a dish or a meal according to the meal plan information, including types and optionally quantities of ingredients.

The term “shortage ingredient information” refers to information indicating ingredients that are required according to the required ingredient information but are not available, or are insufficient, according to the standardized ingredient information from the storage apparatus.

The term “display information” refers to structured data formatted for presentation on a user interface of a portable information terminal, the data including at least meal plan information and shortage ingredient information.

The term “user display interface” refers to a graphical or interactive interface presented on a display of the portable information terminal for presenting information and receiving user input.

The term “order candidate information” refers to information representing a candidate list of items or ingredients to be ordered, including product types and quantities, which the user can edit or confirm based on the shortage ingredient information.

The term “product information management apparatus” refers to an information processing system, such as a server, that manages product-related data including product identification information, availability, and price.

The term “electronic commerce information processing apparatus” refers to an information processing system configured to execute electronic transaction processing, such as order registration, payment processing, and order confirmation, over a communication network.

The term “order information” refers to information transmitted to a commerce-related system to request a purchase, the information including at least product identification information, quantity information, delivery destination information, and desired delivery time information.

The term “product identification information” refers to information uniquely identifying a product within a commerce system, such as a product code, item number, or other identifier.

The term “quantity information” refers to information indicating a requested amount or count of each product to be ordered.

The term “delivery destination information” refers to information that specifies a location to which ordered products are to be delivered, such as an address or delivery point identifier.

The term “desired delivery time information” refers to information indicating a preferred delivery time or delivery time window for ordered products, which may be derived from the meal condition information.

The term “order confirmation information” refers to information returned from a commerce-related system indicating that an order has been accepted, including at least an order identifier or confirmation status.

The term “scheduled delivery information” refers to information indicating a scheduled time or time window for delivery of ordered products.

The term “preference history information” refers to information derived from past user interactions, selections, or feedback, indicating historical user preferences, which can be used to influence or refine subsequent meal plan generation.

The term “update request” refers to a request from the portable information terminal for modification or regeneration of meal plan information previously obtained from the generative AI model.

The term “re-proposal request” refers to a request from the portable information terminal for a new or alternative meal plan distinct from an existing meal plan information set, based at least in part on current ingredient information and meal condition information.

The server, the terminal, and the user cooperate to implement various embodiments of the invention. The server executes a program stored in a memory by using a processor. The terminal executes a client program on a portable information device, such as a smartphone or tablet. The user operates the terminal.

The server uses general-purpose server hardware, such as a multi-core central processing unit, a volatile memory, a non-volatile storage device, and a network interface. The server may run an operating system such as a UNIX-like operating system and a server application framework such as a web application framework. The terminal uses a mobile operating system such as a handheld operating system and includes a touch display, a built-in camera module, a wireless communication interface, and a local storage.

The terminal provides a camera user interface by using a camera application programming interface of the mobile operating system. The terminal uses the built-in image sensor to capture image information of the inside of a storage apparatus such as a refrigerator or freezer. The terminal encodes the captured image into a compressed format such as JPEG and stores the resulting binary data in a local file system. The terminal attaches metadata, such as capture time, image resolution, and device identifier, and stores the metadata in an in-memory data structure.

The terminal provides a graphical user interface to receive meal condition information from the user. The terminal displays input fields for a meal execution time, a cooking required time, a number of persons, and optional conditions such as a nutritional condition, a preference condition, a budget condition, and a cooking time constraint. The terminal converts the meal execution time into a timestamp and converts the additional conditions into key-value pairs. The terminal stores the meal condition information in a structured format, such as a JSON-like object in memory.

The terminal sends the image information and the meal condition information to the server via a communication network. The terminal uses a secure communication protocol such as HTTPS over TCP/IP and an application-layer protocol such as HTTP. The terminal encapsulates the image information as multipart binary data and the meal condition information as a structured payload. The terminal transmits the payload to a designated network address of the server.

The server receives the transmitted data through the network interface. The server uses a web application framework to parse the incoming request. The server separates the image information from the meal condition information and temporarily stores the image file in a storage device, such as a block storage or object storage. The server records in a database an entry that associates a user identifier, an image file path, and the meal condition information.

The server uses a relational database management system or a similar data store. The server defines a schema that includes fields for standardized ingredient information, meal plan information, shortage ingredient information, and order candidate information.

The server executes an image recognition program on the image information. In one embodiment, the server includes an image recognition module implemented as a convolutional neural network. The server uses a convolutional neural network model comprising multiple convolutional layers, pooling layers, batch-normalization layers, and fully connected layers. The server applies the convolutional neural network to the image information to obtain feature maps and classification outputs corresponding to ingredient categories such as “meat,” “vegetable,” “dairy,” or more specific ingredient classes. The server may alternatively or additionally call an external image recognition service through an application programming interface. The server sends the encoded image to the external image recognition service and receives label information and confidence scores.

The server maps raw labels to standardized ingredient information. The server loads a mapping table from the database. The mapping table associates many kinds of raw label strings with canonical ingredient names. The server executes a normalization process that performs steps such as case normalization, synonym lookup, and category mapping. The server groups labels by canonical ingredient name and estimates approximate quantities by examining bounding box sizes or detection counts. The server stores the standardized ingredient information as structured data. The structured data includes a list of ingredient records, each record including a canonical name, a category, an estimated quantity, and a confidence score.

The server retrieves the meal condition information from the database and combines it with the standardized ingredient information to generate a prompt sentence for a generative AI model. The server uses template information stored in the memory. The template information includes text patterns with placeholders for ingredient lists, meal execution times, and constraint expressions. The server uses a character string generation algorithm to fill the placeholders. The algorithm creates a human-readable list of ingredients, converts timestamps into natural-language times, and concatenates constraint clauses.

The server, for example, generates a prompt sentence such as:

“There is chicken, broccoli, and carrots in the refrigerator. I want to prepare dinner by 6:00 PM today for two adults and one child. Please propose several meal plans and recipes that mainly use these ingredients.”

The server may generate another prompt sentence such as:

“There is tofu, mushrooms, spinach, and milk in the refrigerator. I want a vegetarian dinner to be ready by 6:30 PM. Please suggest several healthy meal plans under 600 kcal per person, and list any additional ingredients I need to buy, with approximate quantities.”

The server may generate a prompt sentence including more constraints such as:

“There is pork, cabbage, onions, and potatoes in the refrigerator. I need a gluten-free dinner for 4 people ready by 8:00 PM. My budget for extra ingredients is 1500 yen. Please propose 2-3 meal plans, list the full ingredient lists with quantities, mark which ingredients I already have, and summarize the missing ingredients with estimated cost.”

The server transmits the generated prompt sentence to a generative AI model. In one embodiment, the server communicates with an external generative information processing model through a web-based application programming interface. The server sends a request message containing the prompt sentence and configuration parameters such as a maximum token length, a temperature parameter, and a top probability parameter. The server receives a response message containing generated text.

The server uses a generative AI model that is implemented as a neural network, such as a transformer-based architecture. The generative AI model includes a plurality of encoder and decoder layers, or a sequence of self-attention layers, feed-forward layers, and normalization layers. The model is pre-trained on large-scale text data and fine-tuned on recipe and meal planning data. The model receives tokenized prompt sentences as input. The model represents each token as a vector embedding. The model computes self-attention scores between tokens and applies linear transformations and non-linear activation functions to generate hidden representations. The model then generates output token sequences that form the meal plan information.

The server configures the generative AI model with parameters such as the number of layers, the number of attention heads, the dimensionality of embeddings, and the vocabulary size.

The server or a separate training system pre-trains the model by minimizing a loss function such as cross-entropy between predicted tokens and target tokens. The training system uses an optimization algorithm such as stochastic gradient descent with variants like Adam. The training system performs weight updates by computing gradients of the loss function with respect to model parameters and applying parameter updates with a learning rate schedule.

The training system may apply data augmentation techniques, such as paraphrasing or ingredient substitution, to increase the robustness of the model in recipe-related contexts.

The server, when using such a model, controls input formatting, tokenization, and decoding strategies. The server uses a tokenizer to convert the prompt sentence into tokens and to reconstruct the response into natural-language text. The server selects decoding strategies, such as greedy decoding, beam search, or nucleus sampling, based on desired diversity and determinism. The server thereby controls the operation of the generative AI model in a technical manner that affects the quality, consistency, and computational efficiency of the resulting meal plan information.

The server parses the response information to extract meal plan information. The server applies parsing rules that expect a particular structure, such as a list of dishes, each with a name, an ingredient list, and step-by-step instructions. The server may instruct the generative AI model through the prompt sentence to output a format with separators or labels to ease parsing. The server splits the response text into sections and identifies dish name information, step information, and required ingredient information. The server converts this information into structured data with defined fields.

The server compares the required ingredient information with the standardized ingredient information from the storage apparatus. The server performs set operations between lists of ingredients. The server computes the difference set between required ingredients and available ingredients. The server calculates shortage ingredient information, which indicates missing ingredients and possibly insufficient quantities. The server may compute a shortage quantity by subtracting an available estimated quantity from a required quantity. The server stores the shortage ingredient information in the database.

The server generates display information that includes the meal plan information and the shortage ingredient information. The server formats the display information as structured data for the terminal. The display information includes, for each meal plan, fields such as dish names, ingredient lists with availability flags, and cooking steps. The server marks each ingredient as already available or missing. The server transmits the display information to the terminal using a communication protocol.

The terminal receives the display information and renders a user display interface. The terminal shows the meal plan information and the shortage ingredient information in a structured layout. The terminal allows the user to browse different meal plans, read cooking steps, and review which ingredients are missing. The terminal provides selection controls for the user to mark which meal plans to adopt and which missing ingredients to purchase. The terminal allows the user to edit a list of order candidate information, such as adding or removing items, changing quantities, or selecting alternative ingredients.

The terminal sends the order candidate information to the server. The server receives the order candidate information and associates it with the corresponding shortage ingredient information and user identifier. The server accesses a product information management apparatus or an electronic commerce information processing apparatus. The server transforms the shortage ingredient information and the order candidate information into product identification information and quantity information by referencing a product catalog. The server builds order information including product identifiers, quantities, a delivery destination, and desired delivery time information derived from the meal execution time and cooking required time.

The server transmits the order information to the commerce-related apparatus using a secure communication protocol. The commerce-related apparatus responds with order confirmation information and scheduled delivery information. The server stores the order confirmation information and scheduled delivery information in the database and notifies the terminal. The terminal presents an order status display to the user.

The terminal may send an update request or a re-proposal request when the user desires a modification. The server receives such a request and optionally updates the meal condition information or retrieves preference history information. The server generates a new prompt sentence that includes information about past preferences or user feedback, in addition to the standardized ingredient information and current meal condition information. The server sends the new prompt sentence to the generative AI model and obtains updated meal plan information. The server processes the updated meal plan information in the same manner and provides refined results to the terminal.

The system produces technical effects beyond simple automation of human tasks. The server, by converting raw image information into standardized ingredient information using a structured database schema and an image recognition neural network, increases the accuracy and consistency of ingredient detection compared to manual inspection. The standardized representation allows efficient set operations and comparisons, reducing computation time and memory overhead when dealing with large numbers of ingredients and meal plans.

The server, by using template-based prompt sentence generation and controlled formatting instructions, reduces variability in prompts and stabilizes the behavior of the generative AI model. This improves reproducibility of outputs and reduces the need for repeated trial-and-error interactions. The server manages tokenization, batching, and parameter configuration to reduce the number of network calls and to increase throughput when serving multiple users, thereby reducing communication load.

The server leverages a neural network model architecture and training methodology to generate meal plan information that cannot be feasibly created with simple rule-based systems or manual written recipes in real time. The model internally learns complex dependencies between ingredients, cooking methods, timing constraints, and user preferences. The server exploits these learned representations to generate context-appropriate meal plans under many different combinations of constraints. This allows the system to achieve high-quality meal suggestions at scale with low latency on the server-side hardware.

The server, by integrating meal plan generation with shortage ingredient computation and order formation in a single processing pipeline, avoids redundant data conversions and eliminates intermediate manual steps. The server operates directly on structured data representations and applies algorithms for matching and mapping ingredients to products.

This integrated approach reduces the likelihood of errors, such as missing necessary items or ordering incorrect quantities, and also improves the overall processing speed.

The system adopts specific data structures for meal plan information, ingredient lists, and orders, which allows efficient indexing and querying within the database. The server can quickly retrieve all meal plans associated with a particular user, ingredient, or time constraint.

The use of normalized schemas and consistent identifiers facilitates caching and concurrency control, which further improves performance under heavy usage.

The system also exhibits robustness and adaptability. The server can accept multiple image inputs over time, track changes in available ingredients, and adapt prompt sentences accordingly. The preference history information allows the server to adjust prompt sentences and weight factors when generating new meal plans. For example, the server may log which generated dishes the user frequently selects and use these logs as input features for the generative AI model or for a separate preference-ranking module.

The server and the terminal together implement technical measures that reduce communication load. The server transmits only structured summaries rather than raw neural network outputs. The terminal caches static display components and requests updates only when underlying data changes. The terminal may compress payloads before transmission, and the server may support differential updates that send only updated fields. These techniques lower bandwidth usage and reduce latency.

The invention is not limited to specific hardware or software products. The server may be implemented on various types of computing environments, including on-premise servers, cloud platforms, or edge computing nodes. The terminal may be a smartphone, a tablet, a smart display, or another portable information terminal. The image recognition program and the generative AI model may be implemented using various frameworks and architectures, as long as they perform the described functions of extracting ingredient information and generating meal plan information from a prompt sentence.

In another embodiment, the server may execute both the image recognition model and the generative AI model locally without relying on external services. In yet another embodiment, the server may offload part of the computation to a specialized accelerator, such as a graphics processing unit, a tensor processing unit, or a dedicated neural network accelerator. These variations still embody the same technical concept of transforming image-based ingredient data and user constraints into structured meal plan information and order information through a specific sequence of data processing steps.

In this manner, the server, the terminal, and the user cooperate in a system that implements concrete data processing, neural network-based inference, structured representation, and communication control. The system improves computer technology itself by enabling more accurate ingredient recognition, more efficient prompt sentence preparation, more precise and flexible meal plan generation, and more reliable and automated order handling, all performed with specific data structures, algorithms, and neural network operations that differ from and exceed simple automation of human mental tasks.

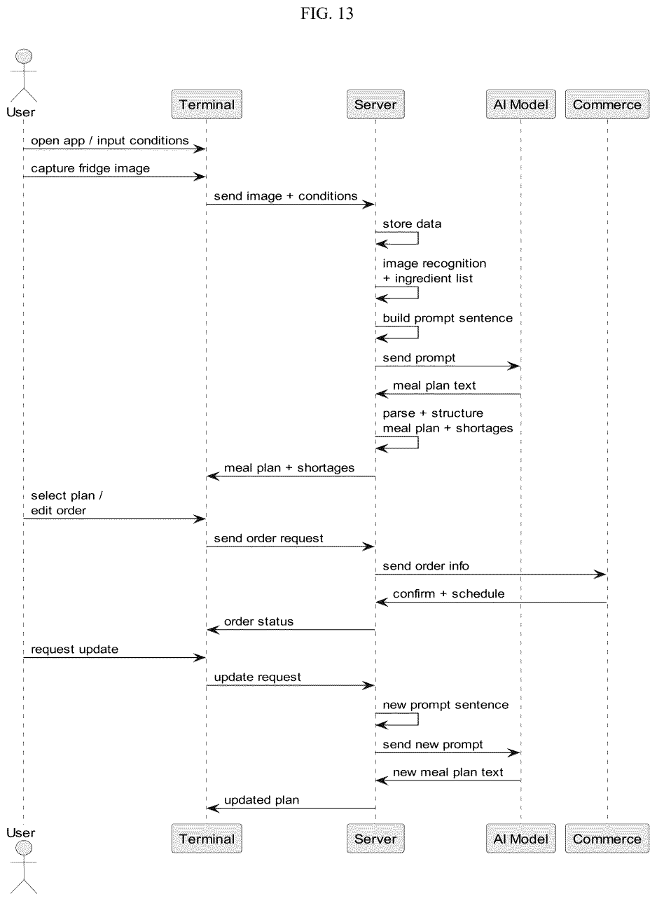

The following describes the processing flow using FIG. 13.

### Step 1:

The user launches an application on the terminal. The input is the user's intent to start meal planning. The terminal loads interface resources from local storage, initializes an in-memory state, and displays a home screen. The output is an active application state ready to accept further user operations.

### Step 2:

The user instructs the terminal to capture an image of a storage apparatus. The input is the user's touch operation on a camera button on the display. The terminal activates a camera module through a camera API, receives raw pixel data from the image sensor, encodes the data into a compressed image format such as JPEG, and writes the binary data to local storage with an associated file path and timestamp. The output is stored image data and metadata identifying that image.

### Step 3:

The user provides meal condition information to the terminal. The input is user operations such as selecting a time from a time picker, entering a number of persons, and toggling preference and budget options. The terminal converts these operations into machine-readable values, such as a timestamp, integers, and enumerated types, and aggregates them into a structured object in memory. The output is structured meal condition information that the terminal can transmit.

### Step 4:

The terminal verifies connectivity and prepares a request to the server. The input is the stored image data and the structured meal condition information. The terminal checks network status via an operating system network API, optionally resizes or compresses the image using an image processing library, and constructs an HTTPS request with a multipart body containing the image and a structured body containing the meal conditions. The output is a formatted network request ready for transmission.

### Step 5:

The terminal sends the image information and meal condition information to the server. The input is the prepared HTTPS request. The terminal uses an HTTP client module to transmit the request over a communication network to a designated endpoint on the server. The output is a network message that reaches the server and contains all required data for further processing.

### Step 6:

The server receives and stores the incoming data. The input is the HTTPS request from the terminal containing image binary data and meal condition fields. The server parses the request using a web framework, separates the image stream from the structured fields, writes the image file to a storage device, and inserts a record into a database table with references to the image path, the user identifier, and the meal condition information. The output is a persistent database entry that uniquely identifies the processing instance.

### Step 7:

The server performs image recognition to detect ingredients. The input is the stored image file referenced by the database entry. The server loads the image into memory and forwards the pixel data to an image recognition module, such as a convolutional neural network or an external image recognition API. The module generates feature representations and classification outputs for objects in the image. The server collects detected labels and confidence scores. The output is a raw ingredient detection result containing candidate ingredient labels and associated scores.

### Step 8:

The server normalizes ingredient information into standardized ingredient data. The input is the raw ingredient detection result from the image recognition step. The server loads a normalization table from the database, maps each raw label to a canonical ingredient name, merges synonyms, and removes low-confidence labels based on threshold comparisons. The server computes approximate quantities or counts when detection data include bounding boxes or multiple instances. The server then assembles a structured list of ingredient records with canonical names, categories, quantities, and confidence values. The output is standardized ingredient information stored in a defined structured format.

### Step 9:

The server retrieves meal condition information and prepares ingredients for text generation.

The input is the standardized ingredient information and the meal condition information associated with the same database entry. The server queries the database to load these items into memory, filters ingredients by confidence threshold, and formats ingredient names into a sequence suitable for natural-language listing. The output is a set of formatted ingredient strings and normalized condition values that can be inserted into a prompt sentence.

### Step 10:

The server generates a prompt sentence for a generative AI model. The input is the formatted ingredient strings and normalized meal condition values. The server selects template information corresponding to the type of meal (for example, dinner) and uses a character string generation algorithm to insert ingredient lists, time expressions, and constraints into textual placeholders. The server performs string concatenation, punctuation handling, and grammatical adjustments. The output is a complete prompt sentence that encodes the ingredients and conditions in natural language.

### Step 11:

The server sends the prompt sentence to a generative AI model and requests meal plan generation. The input is the generated prompt sentence and configuration parameters such as maximum token length and diversity parameters. The server tokenizes the prompt, constructs a request for a transformer-based generative model, and transmits the request using a client library over a network or an internal bus. The server triggers model inference on hardware such as a central processing unit or an accelerator. The output is a generated text response containing proposed meal plans, including dish descriptions, ingredient lists, and cooking steps.

### Step 12:

The server parses the generated text into structured meal plan information. The input is the textual response from the generative AI model. The server applies parsing logic that may use separators, headings, or key markers previously requested in the prompt. The server splits the text into segments, identifies dish names, extracts lines listing required ingredients, and enumerates stepwise cooking instructions. The server converts these segments into structured records and writes them into the database as meal plan entries linked to the original processing instance. The output is structured meal plan information suitable for algorithmic comparison with available ingredients.

### Step 13:

The server computes shortage ingredient information by comparing required and available ingredients. The input is the structured meal plan information and the standardized ingredient information detected from the storage apparatus. The server performs set and multiset operations to identify ingredients present in required lists but absent or insufficient in the available list. The server calculates shortage quantities by subtracting available estimates from required quantities when both are present. The server records, for each meal plan, which ingredients are available and which are missing. The output is shortage ingredient information linked to each meal plan.

### Step 14:

The server constructs display information for the terminal. The input is the meal plan information and the shortage ingredient information. The server organizes meal plans into a hierarchical data structure, such as a list of plan objects each containing dish name information, an annotated ingredient list with availability flags, and ordered step information.

The server formats this structure into a compact representation suitable for transmission, such as a serialized object with defined fields. The output is display information that the terminal can use to render screens for the user.

### Step 15:

The server sends the display information to the terminal. The input is the structured display information object. The server embeds the display information in a response message to a pending request or a push notification message, and transmits it via a communication network using protocols such as HTTPS or a push notification service. The output is a network message that reaches the terminal and delivers all data necessary for user presentation.

### Step 16:

The terminal receives and renders the meal plans and shortage information. The input is the display information transmitted by the server. The terminal parses the structured data into in-memory objects, maps each field to user interface components, and draws list views, text fields, and icons on the display. The terminal indicates, for each ingredient, whether it is already available or missing. The output is a visible screen through which the user can inspect meal options and missing ingredients.

### Step 17:

The user reviews the proposed meal plans and edits order candidate information. The input is the displayed meal plans and shortage ingredient lists. The user selects one or more meal plans, checks the missing ingredients, and performs operations such as selecting items to purchase or adjusting quantities via the terminal's user interface. The terminal translates these operations into a structured representation of order candidate information. The output is order candidate information stored in the terminal's memory and ready for transmission.

### Step 18:

The terminal sends the order candidate information to the server. The input is the structured order candidate information edited or confirmed by the user. The terminal wraps the order candidate information in an HTTPS request message and transmits it to the server. The output is a network message reaching the server containing all required data for order formation.

### Step 19:

The server transforms order candidate information into order information for a commerce system. The input is the order candidate information and the shortage ingredient information stored in the database. The server resolves each ingredient to product identification information using a product catalog table or a commerce system API, attaches quantity information specified by the user, and adds delivery destination information and desired delivery time information derived from the meal execution time and cooking time. The server assembles these elements into an order information object. The output is a complete order information structure suitable for submission to an electronic commerce information processing apparatus.

### Step 20:

The server submits the order information and handles confirmation. The input is the order information. The server sends the order information to an external commerce system using a defined application programming interface and waits for a response. The commerce system returns order confirmation information and scheduled delivery information. The server parses this response, stores the confirmation and schedule in the database, and prepares a status update message for the terminal. The output is persisted order status data and a status notification message.

### Step 21:

The server notifies the terminal of the order status. The input is the order confirmation information and scheduled delivery information stored in the server. The server composes a concise status payload and transmits it to the terminal via HTTPS or a push notification channel. The output is a delivered status update that describes whether the order is accepted and when delivery is planned.

### Step 22:

The terminal displays the order status and delivery schedule. The input is the status update from the server. The terminal parses the status payload, updates local state, and redraws relevant user interface elements to show confirmation identifiers, delivery times, and progress indicators. The output is a current status view that informs the user of the state of ingredient procurement.

### Step 23:

The user optionally requests an update or re-proposal of meal plans. The input is the displayed meal plans and order status. The user may operate a control on the terminal to indicate dissatisfaction, change of preference, or a need for alternative menus. The terminal converts this operation into an update request or re-proposal request, optionally attaching feedback such as disliked ingredients or desired cuisines. The output is a structured request sent from the terminal to the server.

### Step 24:

The server regenerates a prompt sentence reflecting updated conditions and preferences. The input is the update or re-proposal request, the standardized ingredient information, the meal condition information, and preference history information from the database. The server adjusts constraint values or introduces additional preference terms, selects an appropriate template, and uses the character string generation algorithm to produce a new prompt sentence. The server incorporates indication of past user selections and feedback as explicit instructions or soft constraints in the text. The output is a refined prompt sentence that better matches the user's history and current situation.

### Step 25:

The server re-queries the generative AI model and updates meal plan information. The input is the refined prompt sentence and model configuration parameters. The server executes a second inference call to the generative AI model, receives new generated text, and repeats parsing and structuring operations as in previous steps. The server replaces or augments previous meal plan records in the database and transmits corresponding updated display information to the terminal. The output is updated meal plan information reflecting the user's feedback and revised conditions.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

Conventional computer-implemented recipe and shopping assistants typically perform only rule-based filtering or template-based menu generation on the basis of static ingredient lists and simple user preferences. Such systems exhibit several technical deficiencies at the computing-system level.

First, conventional systems are not architected to robustly integrate heterogeneous sensor streams and high-dimensional inference outputs such as image-based refrigerator inventory, user schedule parameters, budget constraints, and emotion-related signals into a single coherent representation that can be consumed by a generative AI model. As a result, each subsystem (image recognition, scheduling, budgeting, emotional analysis) often operates in isolation, and the processor cannot efficiently coordinate these heterogeneous data to produce context-appropriate prompts. This leads to repeated network calls, redundant data conversions, and increased processor load, thereby degrading overall system throughput and response time.

Second, in existing approaches the construction of input for a generative AI model is typically static or manually defined. A processor may simply concatenate text fields without explicit encoding of constraints and without adaptation to the current state of refrigerator inventory, the user's temporal constraints, or the user's emotional state. This naive prompt construction causes the generative AI model to generate plans that are inconsistent with real-world constraints, which in turn forces additional rounds of post-processing, re-prompting, and user correction. This loop increases computational overhead, network traffic, and storage operations in the computing environment.

Third, conventional systems lack an integrated control flow that automatically propagates the generative AI output into downstream machine-to-machine interactions such as price retrieval, stock checking, order optimization, and delivery scheduling. Even if a meal plan is generated, the processor is not configured to algorithmically compare required ingredients with current inventory, compute optimized candidate purchase combinations under budget constraints, and trigger ordering and delivery workflows without further user-driven orchestration. This absence of an end-to-end control mechanism results in fragmented processing, duplicated business logic across components, and inefficient utilization of communication resources.

Fourth, while emotion recognition technologies and recommender systems are known individually, conventional architectures do not technically couple emotion analysis results into the core planning pipeline at the level of prompt construction, constraint selection, and complexity adjustment of cooking instructions. As a consequence, existing systems do not adapt computational behavior such as recipe complexity selection or the structure of generated instructions to the user's current emotional state. This yields suboptimal task planning and user interactions, which in turn increases the number of user corrections and re-requests, and thereby increases processor cycles and network load.

Accordingly, there is a need for an improved computer-implemented system in which a processor is specifically configured to: (i) acquire and structure refrigerator inventory from image data, (ii) collect and normalize usage conditions and emotion-related data, (iii) algorithmically generate constraint-aware prompt sentences for a generative AI model, (iv) parse and structurally store generated meal plans, and (v) automatically derive and execute optimized purchasing and delivery actions. By tightly integrating these functions, the system can reduce unnecessary re-prompting, minimize redundant data processing, and improve the technical performance of the overall computing environment in terms of latency, resource utilization, and robustness of end-to-end meal planning operations.

The specific processing by the specific processing unit 290 of the data processing device 12 in Application Example 2 is realized by the following means.

The present invention provides a server comprising a processor configured to acquire state information of a storage appliance including an interior of a cooling compartment and to process image data indicating the interior of the storage appliance by using image analysis technology in order to identify stored ingredient information and store the ingredient information in a structured form in a storage device; to provide, via a user terminal, an information input and output interface through which a user inputs usage conditions including at least one of a meal provision time, a cooking time, a shopping-available time, a menu provision period, a menu provision frequency, budget information, and preference information, and to store the usage conditions in association with a user identifier; to acquire emotion-related input data or biometric information from which an emotional state of the user is inferable, to determine the emotional state of the user by using emotion analysis technology, and to store the emotional state in association with the user identifier; to generate, on the basis of the structured ingredient information, the stored usage conditions, and the determined emotional state, a prompt sentence that explicitly encodes constraints for input to a generative AI model; to transmit the prompt sentence to the generative AI model via a communication network and to receive from the generative AI model a meal plan including at least a menu name, required ingredients, required quantities, and cooking procedures that satisfy the constraints; to parse the received meal plan into structured plan data and store the structured plan data; to compare the required ingredients included in the structured plan data with the stored ingredient information to identify missing ingredients; to obtain, via the communication network and from a merchandise information providing apparatus, price information and stock information for the missing ingredients, to calculate and propose combinations of candidate ingredients that are purchasable within the budget information, and to automatically execute purchase processing for the candidate ingredients based on a selection by the user or a preset condition; and to cooperate with a delivery management apparatus such that, for the candidate ingredients for which the purchase processing has been completed, delivery processing is automatically executed so that the candidate ingredients are delivered at a delivery time designated by the user or at a delivery time corresponding to the meal provision time. This enables the computing system to automatically integrate multi-modal sensor data, user constraints, and emotion analysis into a single generative-AI-driven planning pipeline, thereby reducing redundant network transactions and re-computation, improving prompt relevance and generative model output quality, and increasing overall processing efficiency and responsiveness in generating, materializing, and adapting meal plans.

The term “storage appliance” refers to a physical apparatus configured to store food items under controlled environmental conditions, including but not limited to a refrigerator, freezer, or similar cooling device having an interior compartment.

The term “cooling compartment” refers to an interior space of a storage appliance in which temperature is controlled to preserve food items, and which can be imaged by an imaging device.

The term “image data” refers to digital data representing a still image or sequence of images captured by an imaging device, the data including pixel values or compressed representations suitable for processing by image analysis technology.

The term “image analysis technology” refers to a software-implemented technique or algorithm that processes image data to detect, classify, or localize objects, including but not limited to computer vision models, pattern recognition algorithms, and machine-learning-based image recognition.

The term “ingredient information” refers to structured information indicating at least a type and optionally a quantity of a food item stored in the storage appliance.

The term “structured form” refers to a machine-readable data representation, such as a record, list, table, or object, in which elements of information are organized into predefined fields or attributes.

The term “storage device” refers to a hardware component or subsystem configured to store data, including but not limited to a magnetic disk, solid-state drive, memory device, or a network-accessible storage service.

The term “user terminal” refers to an information processing device operated by a user, including but not limited to a smartphone, tablet, personal computer, or dedicated terminal, capable of communicating with the server via a communication network.

The term “information input and output interface” refers to a logical or graphical interface provided on a user terminal through which a user can input data and receive outputs, such as a graphical user interface with text fields, buttons, and display areas.

The term “usage conditions” refers to constraint information provided by a user and used for planning, the information including at least one of a meal provision time, a cooking time, a shopping-available time, a menu provision period, a menu provision frequency, budget information, and preference information.

The term “meal provision time” refers to a point in time or time interval at which a meal is intended to be served or completed.

The term “cooking time” refers to an allowable time duration within which preparation and cooking of a meal are to be completed.

The term “shopping-available time” refers to a point in time or time interval during which a user is able to perform or receive shopping, either physically or via delivery.

The term “menu provision period” refers to a span of calendar time over which meal plans or menus are to be provided to the user.

The term “menu provision frequency” refers to the number of meals or menus to be provided within a given menu provision period.

The term “budget information” refers to monetary constraint data indicating a maximum amount of expenditure permitted for purchasing ingredients or related items.

The term “preference information” refers to user-specific data indicating likes, dislikes, dietary restrictions, cuisine preferences, or other qualitative conditions relevant to meal planning.

The term “user identifier” refers to data that uniquely associates information with a specific user, such as an ID, token, or account reference.

The term “emotion-related input data” refers to data explicitly or implicitly expressing a user's emotional state, including but not limited to self-reported text, selection of an emotion label, or other user-provided information indicative of mood.

The term “biometric information” refers to sensor-derived data relating to physical or behavioral characteristics of a user, including but not limited to facial images, voice signals, and other physiological signals that can be used to infer an emotional state.

The term “emotional state” refers to a categorized or quantified representation of a user's current emotional condition, such as stress, joy, sadness, tiredness, or excitement, as determined by emotion analysis technology.

The term “emotion analysis technology” refers to a software-implemented technique that processes emotion-related input data or biometric information to infer or classify an emotional state, including but not limited to machine-learning-based emotion recognition algorithms.

The term “generative AI model” refers to a machine-implemented model that generates output data, such as text describing a meal plan, in response to input data, and that is trained using machine learning techniques on large amounts of data.

The term “prompt sentence” refers to a text sequence or structured textual input generated for submission to a generative AI model, the text encoding a task description and constraints for the generative AI model.

The term “constraints” refers to conditions encoded in a prompt sentence that the generative AI model is expected to satisfy, including but not limited to ingredient availability, time limitations, budget limitations, and emotion-related requirements.

The term “meal plan” refers to information describing at least one meal, including a menu name, required ingredients, required quantities, and cooking procedures.

The term “menu name” refers to a textual designation identifying a particular dish or meal.

The term “required ingredients” refers to a set of food items specified by type that are needed to prepare a menu included in a meal plan.

The term “required quantities” refers to numerical amounts or units associated with required ingredients in a meal plan.

The term “cooking procedures” refers to ordered instructions or steps describing operations to be performed to prepare a meal.

The term “structured plan data” refers to a machine-readable representation of a meal plan in which items such as menu names, required ingredients, required quantities, and cooking procedures are stored in defined data fields or records.

The term “missing ingredients” refers to those ingredients that are required by a meal plan but are absent from, or present in insufficient quantities in, the stored ingredient information.

The term “communication network” refers to a wired or wireless data transmission infrastructure enabling data exchange between the server, the user terminal, and external apparatuses.

The term “merchandise information providing apparatus” refers to an external information processing system that supplies data related to merchandise, including price information and stock information for ingredients or products, via a communication interface.

The term “price information” refers to data indicating monetary cost for a unit or package of an ingredient or product.

The term “stock information” refers to data indicating availability and quantity of an ingredient or product in an inventory of a supplier.

The term “candidate ingredients” refers to one or more specific purchasable products or product combinations selected as satisfying a missing ingredient requirement and being within a user's budget.

The term “purchase processing” refers to a series of operations executed by the server to complete an order for candidate ingredients, including selection, order creation, payment initiation, and transmission of order data to a supplier.

The term “preset condition” refers to configuration information predetermined by the user or system, specifying circumstances under which the server is permitted to automatically execute purchase processing without explicit user confirmation.

The term “delivery management apparatus” refers to an external information processing system that manages logistics and delivery scheduling for products ordered by the server.

The term “delivery processing” refers to operations performed to schedule and control shipment of ingredients, including generation of delivery instructions, assignment of delivery times, and communication with logistics resources.

The term “delivery time” refers to a time or time window at which ordered ingredients are to be delivered to a location associated with the user.

The term “selection by the user” refers to an operation performed through the user terminal in which a user explicitly chooses or confirms one or more candidate ingredients, meal plans, or options presented by the system.

In one or more embodiments, a server cooperates with at least one user terminal and at least one storage appliance such as a refrigerator to implement the claimed system. The server includes at least one processor, a main memory, a non-volatile storage device, and a network interface coupled via a bus. The terminal includes a processor, a memory, an imaging device such as a camera, a display, an input device, and a communication interface.

The storage appliance includes at least one imaging sensor and optionally additional sensors such as weight sensors or identification tags.

In a representative embodiment, the server executes a program stored in the non-volatile storage device and loaded into the main memory. The program defines software modules including an image acquisition and analysis module, an inventory management module, a condition management module, an emotion analysis module, a prompt generation module, a generative AI client module, a plan parsing and storage module, a comparison and shopping module, and a delivery coordination module. The modules communicate via defined data structures stored in a database system, such as a relational database or a key-value store.

The server uses general-purpose computing hardware, for example a virtual machine instance on a cloud computing platform, and can execute operating system software such as a general-purpose server operating system. The terminal can be implemented as a smartphone, tablet, or personal computer running a mobile operating system or a desktop operating system. The terminal executes an application providing a graphical user interface for capturing images, entering usage conditions, confirming purchases, and viewing meal plans and cooking procedures.

The server uses image analysis technology implemented as a convolutional neural network-based image recognition model. In one embodiment, the server uses a deep neural network similar to those provided by cloud-based image recognition services, including multiple convolutional layers, pooling layers, and fully connected layers. The image recognition model receives image data captured by a camera inside the cooling compartment or by the terminal and outputs, for each detected region, a label representing a food item and a confidence score. The server stores the labels and associated quantities in a structured inventory table keyed by a user identifier and a timestamp.

The terminal uses its camera to capture image data in a standard format such as JPEG. The terminal pre-processes the image data by resizing, compressing, and attaching metadata such as the user's account identifier and a capture time. The terminal then transmits the image data to the server over a communication network using a secure protocol. The server receives the image data and stores it in a binary large object storage. The image acquisition and analysis module of the server then retrieves the stored image and passes it to the image recognition model. The model extracts feature maps by convolving learned filters over pixel values and computes classification probabilities for candidate ingredient categories. The server uses predefined thresholds and a mapping table to convert the model outputs into standardized ingredient identifiers.

The server uses an inventory management module to aggregate multiple recognition results.

The server converts detected units into normalized quantities, for example by associating bounding box sizes with standard counts or by summing multiple detections for the same category. The server stores the aggregated inventory as records each including an ingredient identifier, a normalized quantity, a unit, and a freshness or timestamp attribute. This structured representation enables subsequent comparison operations to be executed as simple database queries rather than repeated image recognition.

The terminal provides an information input and output interface implemented as a graphical user interface. The terminal displays input controls such as text fields, sliders, dropdown menus, and date and time pickers. The user uses the terminal to enter usage conditions such as a meal provision time, an available cooking time, a shopping-available time, a menu provision period and frequency, a budget, and food preferences. The terminal packages these usage conditions into a structured message and transmits the message to the server. The condition management module of the server stores the received usage conditions in a normalized format, associating them with the user identifier.

The server optionally uses an emotion analysis module to determine an emotional state of the user. The terminal can acquire emotion-related input data in multiple ways. In one embodiment, the user selects a current mood from a list (for example, “stressed”, “tired”, “happy”, “excited”), and the terminal forwards the selected label to the server. In another embodiment, the terminal uses the camera and microphone to capture facial images or voice samples. The terminal transmits these sensor data to the server. The server processes the sensor data using an emotion analysis technology implemented as a neural network classifier.

The classifier can be realized as a deep neural network with convolutional layers for facial images or recurrent layers for audio sequences, trained to output probabilities for multiple emotion classes. The server selects an emotional state label and a confidence score and stores this information in association with the user identifier and current session.

The server uses the prompt generation module to construct a prompt sentence for a generative AI model. The module retrieves the current inventory data, the stored usage conditions, and the determined emotional state. The module then creates a text string that explicitly encodes these constraints in a human-readable form. The prompt generation module uses a template system in which ingredient lists, time constraints, budget values, and emotion labels are inserted into predetermined sentence structures. For example, the server can generate a prompt sentence such as:

“There is chicken and cabbage in the refrigerator. Dinner should be ready by 18:00 today.

The user is feeling stressed and prefers easy, comforting food. Please propose one optimal dinner menu, list the required ingredients and their quantities, and provide step-by-step cooking instructions.”

In another example, the server can generate a prompt sentence such as:

“With a budget of 3000 yen, and the following ingredients in the fridge: tofu, eggs, onions, carrots, miso, and soy sauce, please propose Japanese dinner menus for the next 3 days.

Consider that the user is tired and has at most 30 minutes each evening. Output the menus, the required ingredients and their quantities, and the cooking steps in a clearly structured textual format.”

The generative AI client module of the server transmits the prompt sentence to a generative AI model executed on a remote computing resource or on the same server. The generative AI model can be implemented as a large language model including multiple transformer layers, each layer having attention mechanisms that process tokenized representations of the prompt sentence. The model is trained using machine learning on large text corpora including recipes, ingredient lists, and natural language instructions. Training uses a loss function such as cross-entropy between predicted token distributions and ground-truth sequences, and a gradient-based optimization algorithm such as stochastic gradient descent or a variant. During inference, the model processes the prompt sentence to generate a sequence of tokens representing one or more meal plans.

The server uses the plan parsing and storage module to convert the generated text into structured plan data. The module applies pattern recognition rules and optionally a secondary language model constrained to output a limited schema to extract menu names, ingredient lists, quantities, and cooking procedures. The server stores this structured plan data in the database. The use of structured storage allows the server to perform efficient comparisons and aggregations at the database level, reducing the need for additional natural language parsing operations in subsequent steps.

The server uses the comparison and shopping module to identify missing ingredients. The module issues database queries that join the structured plan data with the inventory data on ingredient identifiers. The module computes differences between required quantities and available quantities. For each ingredient, the module marks the ingredient as available, insufficient, or missing. The module constructs a list of missing or insufficient ingredients along with required additional quantities.

The server uses a communication network to query one or more merchandise information providing apparatuses. These apparatuses can be implemented as remote servers operated by retail systems accessible through application programming interfaces. The server sends requests containing product descriptions or standardized ingredient identifiers and receives price information and stock information as structured responses. The server then uses an optimization routine to calculate one or more candidate combinations of products that satisfy the required quantities under the given budget. In one embodiment, the server formulates a constrained optimization problem in which the objective function is to minimize total cost subject to satisfying each ingredient requirement. The server uses a linear or integer programming solver or a greedy approximation algorithm to select candidate products. The server associates each candidate combination with its total cost and estimated delivery availability.

The server then transmits the meal plan, missing ingredients, candidate product combinations, and cooking procedures to the terminal. The terminal displays these elements using the graphical user interface. The user can examine the proposed menus, review which ingredients are already available, and view the additional ingredients and their costs. The user can select a set of candidate ingredients and confirm a purchase. Alternatively, the server can initiate automatic purchase processing based on preset conditions stored in the system configuration, for example when the total cost is under a predefined threshold or when the user previously authorized automatic ordering.

The server uses the purchase processing part of the comparison and shopping module to construct an order payload including product identifiers, quantities, delivery address, payment method information, and requested delivery times. The server transmits the order payload to a delivery management apparatus. The delivery management apparatus performs logistics scheduling and returns confirmation data including order identifiers and planned delivery times. The server stores the confirmation data and notifies the terminal. The user can then view a detailed order confirmation and an expected delivery time. The cooperation between the server and the delivery management apparatus allows the system to automatically connect generative AI-based meal planning with physical world logistics, thereby achieving concrete control over the delivery of ingredients.

The server optionally uses the emotion analysis module and prompt generation module to adapt cooking procedures to the emotional state and the skill level of the user. For example, when the stored emotional state indicates stress or tiredness, the server can generate a secondary prompt sentence such as:

“Here is a recipe for ‘Chicken and Vegetable Stir-Fry’: [insert original steps]. The user is a beginner and is feeling tired. Rewrite the recipe so that it uses as few steps as possible, minimizes washing up, and provides very clear and short instructions.”

The generative AI model processes this secondary prompt and outputs simplified instructions.

The server parses and stores the adapted steps and provides them to the terminal. This adaptive behavior is not a mere automation of human reasoning; the server transforms high-dimensional emotion and inventory data into machine-optimized prompts and structured outputs in a way that is specific to the processing strengths of neural networks and database systems.

In another embodiment, the server uses data from multiple sessions to refine its prompt generation and selection strategies. The server stores historical prompts, generated plans, user selections, and delivery outcomes. A separate analysis process executed by the server can use these data to update parameters that control prompt templates, such as preferred recipe complexity, typical budget usage, and ingredient acceptance. For example, when the server detects that the user repeatedly rejects certain ingredients, the prompt generation module can automatically reduce the weight or remove those ingredients when constructing new prompt sentences. This adaptation reduces the number of unnecessary prompt-response cycles, lowers communication load with the generative AI model, and improves overall computational efficiency.

The described architecture provides a technical improvement over conventional systems. The server does not simply store and display user data; it integrates multiple sensor modalities, performs complex normalization and aggregation operations, and generates constraint-aware prompt sentences that enable the generative AI model to produce outputs already aligned with real-world constraints. This reduces the need for repeated queries and manual corrections, leading to lower processor usage and network traffic. The use of structured plan data and normalized inventory data allows the server to execute comparisons and optimizations using efficient database and optimization algorithms, thereby improving processing speed and scalability. The emotion-aware adaptation of prompt sentences and cooking procedures reduces user interaction steps and re-requests, which further optimizes computational and communication resources.

The terminal and the storage appliance participate in concrete control of physical devices.

The terminal controls the camera to capture images and sensor data at appropriate resolutions and frequencies. The storage appliance can be configured to automatically capture images at intervals or in response to events, such as door openings, and to transmit the images to the server. The server can adjust the capture schedule based on recognition results and user behavior; for example, the server can reduce imaging frequency when inventory changes infrequently, thereby lowering energy use and data volume.

In another embodiment, the server can execute the image recognition model locally instead of using a remote service. The server can preload a trained convolutional neural network model into memory. The model can be compressed or quantized to reduce memory footprint and inference time. The server can apply data augmentation techniques during model training, such as random cropping, rotation, and brightness changes, to increase robustness against variations in lighting and camera angle. These technical measures improve recognition accuracy and stability in the operating environment of domestic refrigerators, thereby reducing misclassification-induced reprocessing.

In yet another embodiment, the generative AI model can be specialized for culinary tasks by fine-tuning on domain-specific data. The server can use a training process in which meal plan outputs are evaluated against constraints such as ingredient availability, budget, and time, and feedback signals are used to adjust model parameters. Although training may be performed off-line, the resulting model embedded in the system exhibits improved alignment between generated meal plans and system constraints. This alignment further reduces downstream adjustments, parsing errors, and wasted computational cycles.

The system is not limited to refrigerators and can be applied to other storage appliances such as pantries, freezers, or vending machines equipped with imaging sensors. The same architecture can support multiple user terminals and multiple storage appliances per user. The server can maintain separate inventories by appliance and merge them logically when constructing prompt sentences. By using a consistent data model and modular processing components, the server can scale to handle many users in parallel while maintaining low latency for each session.

Through these embodiments, the server, terminal, and storage appliance cooperate to realize a concrete implementation of the claimed system. The server uses specific AI model architectures, defined data structures, and non-conventional prompt generation and optimization procedures to achieve technical effects including improved processing speed, reduced error rates in inventory recognition and plan generation, reduced network traffic through fewer iterations with the generative AI model, and more efficient management of structured inventory and plan data. These technical improvements differentiate the system from mere automation of human meal planning and provide a concrete enhancement in the operation of computer technology in the context of generative AI-driven meal planning and ingredient delivery.

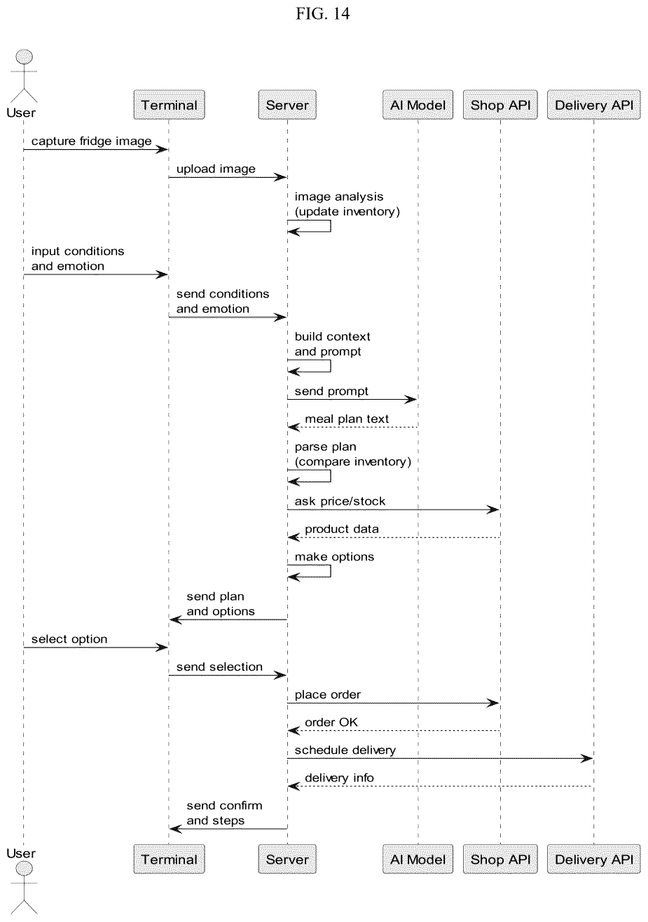

The following describes the processing flow using FIG. 14.

### Step 1:

User operates the terminal to capture an interior image of the storage appliance.

User points the terminal's camera toward the interior of the cooling compartment and activates a capture control on the terminal's application.

Input: light from the interior of the storage appliance received by the camera sensor.

Processing: the terminal converts the optical signal into digital image data, encodes the image in a format such as JPEG, and attaches metadata including a user identifier, a capture timestamp, and an appliance identifier.

Output: encoded image data with associated metadata stored temporarily in the terminal's memory.

### Step 2:

Terminal transmits the captured image data to the server.

Terminal opens a secure communication channel to the server via a network interface and constructs a request message including the encoded image and metadata.

Input: encoded image data and metadata stored in the terminal.

Processing: the terminal creates an HTTP or similar network request, embeds the image data as a payload, and performs error checking such as payload size verification before transmission.

Output: a network message containing the image data and metadata delivered to the server.

### Step 3:

Server stores and pre-processes the received image data.

Server receives the network message, extracts the image payload and metadata, and registers the data in persistent storage.

Input: network message containing encoded image data and metadata.

Processing: the server validates the file type and integrity, assigns an internal image identifier, writes the image to a binary storage area, and inserts a record into an image table linking the internal identifier with the user identifier and timestamp.

Output: a stored image record referenced by an internal image identifier.

### Step 4:

Server executes image analysis to identify ingredient information.

Server retrieves the stored image and passes it to an image analysis module implemented as a convolutional neural network or similar model.

Input: image data referenced by the internal image identifier.

Processing: the server normalizes pixel values, resizes the image if necessary, and feeds the processed image into the image recognition model, which applies convolution, pooling, and classification layers to output labels and confidence scores for detected objects; the server filters out low-confidence detections and maps labels to standardized ingredient identifiers using a mapping table.

Output: a set of ingredient entries each including a standardized ingredient identifier, an estimated quantity indicator, and a confidence score.

### Step 5:

Server normalizes and stores refrigerator inventory data.

Server aggregates ingredient entries from the image analysis and converts them into a structured inventory record.

Input: set of recognized ingredient entries for the current image.

Processing: the server merges entries with identical ingredient identifiers, estimates quantities using heuristics based on bounding box sizes or object counts, converts quantities into normalized units, and attaches freshness or timestamp attributes.

Output: a structured inventory dataset stored in an inventory table associated with the user identifier.

### Step 6:

Terminal acquires usage conditions from the user.

Terminal displays an input interface for the user to specify time and budget constraints and other usage conditions.

Input: user actions such as text inputs, selections, and time picker values.

Processing: the terminal collects values for at least meal provision time, available cooking time, shopping-available time, menu provision period, menu provision frequency, budget, and preference information, validates formats, and aggregates them into a structured data object.

Output: a usage condition object temporarily stored in the terminal.

### Step 7:

Terminal transmits the usage conditions to the server.

Terminal sends the collected usage conditions to the server via the communication network.

Input: usage condition object including all user-specified fields and the user identifier.

Processing: the terminal serializes the usage conditions to a machine-readable format, attaches authentication data, and sends the message to a predefined server endpoint.

Output: a network message containing usage conditions delivered to the server.

### Step 8:

Server stores and normalizes usage conditions.

Server receives the usage conditions and registers them as part of the current planning session.

Input: network message containing usage condition data and user identifier.

Processing: the server validates that all mandatory fields are present, converts times into a uniform time representation, converts budget values into a standard currency unit, and writes the normalized usage conditions into a condition table keyed by a session identifier and user identifier.

Output: stored and normalized usage condition records associated with the user and session.

### Step 9:

Terminal and server acquire emotion-related data.

User provides emotion-related input through the terminal.

Input: user actions such as selecting an emotion label (for example “stressed” or “happy”) or allowing capture of facial image or voice data.

Processing by the terminal: the terminal either encodes the selected label into a short text field or captures image or audio data using local sensors, attaches metadata, and sends the resulting emotion-related message to the server.

Output: a network message containing emotion-related data delivered to the server.

### Step 10:

Server determines the emotional state of the user.

Server uses an emotion analysis module to convert the emotion-related data into an emotional state label and score.

Input: emotion-related data such as a self-reported label, facial image, or audio sample.

### Processing:

when a text label is received, the server directly maps the label to an internal emotional state code;
when sensor data are received, the server pre-processes the data (for example, crops and normalizes face images or extracts audio features), applies a neural network classifier trained on emotion recognition tasks, computes class probabilities, and selects the emotion with highest probability above a threshold.
Output: an emotional state representation including an emotion label and a confidence value stored in an emotion table associated with the user and session.

### Step 11:

Server aggregates inventory, usage conditions, and emotional state.

Server prepares combined context data as a basis for prompt generation.

Input: inventory dataset for the user, normalized usage conditions, and emotional state records for the current session.

Processing: the server retrieves the latest inventory record, retrieves the corresponding usage conditions, retrieves the current emotional state, and combines them into an internal context structure containing ingredient lists, time constraints, budget constraints, preference flags, and an emotional state code.

Output: a unified context data structure held in working memory.

### Step 12:

Server generates a constraint-aware prompt sentence for the generative AI model.

Server converts the context data into a natural language instruction that encodes relevant constraints.

Input: unified context data structure containing ingredients, times, budget, preferences, and emotional state.

Processing: the server selects a prompt template appropriate to the planning scenario (for example, single meal or multi-day plan), inserts ingredient names, time values, budget limits, and emotion descriptions into template fields, and constructs a textual prompt sentence that specifies a task for the generative AI model.

Output: a prompt sentence string ready for transmission to the generative AI model.

### Step 13:

Server transmits the prompt sentence to the generative AI model and receives a textual meal plan.

Server uses a client module to interact with the generative AI model over a network connection or local interface.

Input: prompt sentence string and configuration parameters such as model type, maximum output length, and temperature.

Processing: the server formats the prompt and parameters into a model request, sends the request to the generative AI model, waits for the response, and receives a generated text containing one or more meal plans including menus, ingredients, quantities, and cooking procedures.

Output: generated meal plan text associated with the current session.

### Step 14:

Server parses the generated meal plan into structured plan data.

Server converts the free-form text from the generative AI model into structured records.

Input: generated meal plan text.

Processing: the server applies parsing rules and pattern matching to identify menu names, ingredient lists, and instructions; if needed, the server uses a secondary constrained parser or model to enforce a specific structure, and then populates data fields such as menu identifier, ingredient identifier, required quantity, unit, and ordered steps for cooking.

Output: structured plan data stored in a meal plan table associated with the session and user.

### Step 15:

Server compares required ingredients with current inventory to identify missing ingredients.

Server performs a data comparison between plan requirements and stored inventory.

Input: structured plan data containing required ingredients and quantities; inventory dataset containing available ingredients and quantities.

Processing: the server joins the plan and inventory tables on standardized ingredient identifiers, computes differences between required and available quantities for each ingredient, and flags each ingredient as available, insufficient, or missing depending on the computed difference.

Output: a missing ingredient list including ingredient identifiers and deficit quantities.

### Step 16:

Server queries merchandise information providing apparatuses for price and stock.

Server obtains product data needed to satisfy missing ingredient requirements.

Input: missing ingredient list and budget information from usage conditions.

Processing: the server formulates requests to external merchandise information systems, transmits ingredient descriptions or identifiers, receives price and stock responses, and associates each candidate product with an ingredient identifier and available quantity.

Output: a candidate product dataset containing product identifiers, prices, available stock, and ingredient mappings.

### Step 17:

Server computes candidate ingredient combinations under budget constraints.

Server selects combinations of candidate products that cover all missing ingredients within budget.

Input: candidate product dataset and budget information.

Processing: the server models the selection of products as a constrained optimization problem, computes total costs for various combinations that satisfy required quantities, and uses an optimization algorithm (for example, linear programming or a greedy heuristic) to generate one or more candidate combinations whose total cost does not exceed the budget.

Output: a set of candidate ingredient combinations each including a list of products, quantities, and a total cost.

### Step 18:

Server sends the meal plan and candidate combinations to the terminal.

Server prepares data for user review and confirmation.

Input: structured plan data, missing ingredient list, and candidate ingredient combinations.

Processing: the server converts these data into a format suitable for presentation, such as a structured response containing textual descriptions and numeric values, and transmits the response to the terminal.

Output: a response message containing the meal plan, missing ingredients, and candidate combinations delivered to the terminal.

### Step 19:

Terminal presents the meal plan and purchase options; user selects or confirms a combination.

Terminal displays menus, required ingredients, and purchase options to the user.

Input: response message from the server containing plan and combination data.

Processing by the terminal: the terminal renders menu titles, cooking time estimates, ingredient lists, and combination details such as product names and prices; the user interacts with the interface to select a preferred combination or modify quantities, and the terminal records the user selection.

Output: a user selection message including the chosen candidate combination, or an indication of auto-approval, transmitted back to the server.

### Step 20:

Server executes purchase processing for selected candidate ingredients.

Server performs order creation and submission to external ordering systems.

Input: user selection message indicating a chosen candidate combination and related session data.

Processing: the server builds an order payload that includes product identifiers, quantities, user delivery address, payment information or tokens, and a requested delivery time based on the meal provision time; the server transmits the order payload to an external ordering or delivery system and receives confirmation including order identifiers and delivery schedules.

Output: an order confirmation dataset stored in an order table and a confirmation notification sent to the terminal.

### Step 21:

Server coordinates delivery timing with the delivery management apparatus.

Server ensures that the selected ingredients are delivered at appropriate times.

Input: order confirmation dataset and meal provision time or user-specified delivery time.

Processing: the server transmits scheduling instructions to a delivery management apparatus, specifying desired delivery windows; the delivery management apparatus allocates logistics resources and returns final delivery times, and the server updates order records and informs the terminal.

Output: finalized delivery schedule associated with each order and a status update sent to the terminal.

### Step 22:

Server optionally adapts cooking procedures based on emotional state and user feedback.

Server refines instructions to match user capability and emotional condition.

Input: structured plan data including original cooking procedures, emotional state records, and optionally historical feedback on recipe complexity.

Processing: the server generates a secondary prompt sentence that embeds the original steps and desired adjustments, sends the prompt to the generative AI model, receives modified instructions, parses them into structured steps, and updates the cooking procedures stored for the session.

Output: adapted cooking procedure data sent to the terminal for display.

### Step 23:

Terminal presents adapted cooking procedures and usage guidance to the user.

Terminal guides the user through execution of the meal plan.

Input: adapted cooking procedure data and meal plan details from the server.

Processing: the terminal formats the procedures into step-by-step views, may include timers or progress indicators, and presents them to the user as the user advances through the steps; the terminal may record completion or difficulty feedback.

Output: a sequence of user interactions and optional feedback data that can be transmitted back to the server for future optimization.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

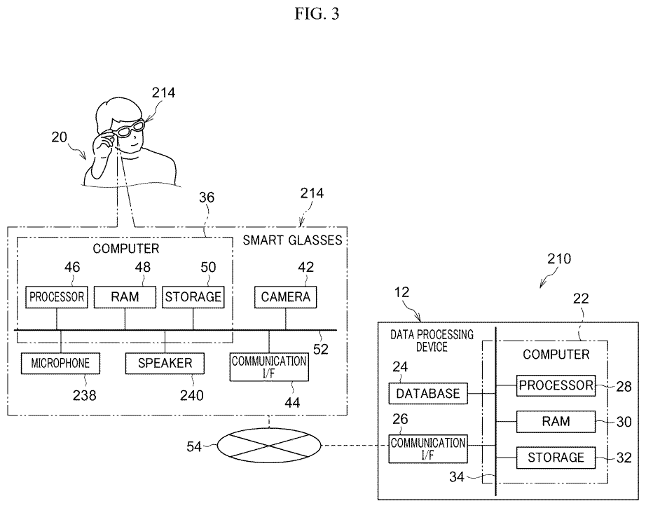

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

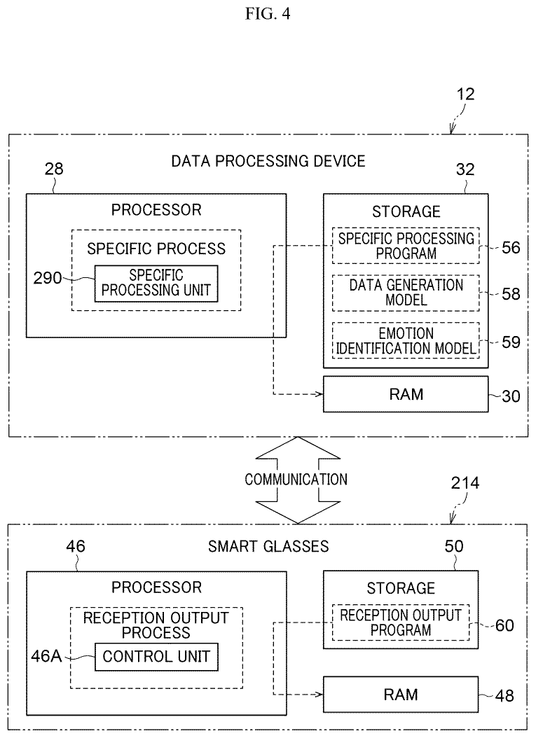

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

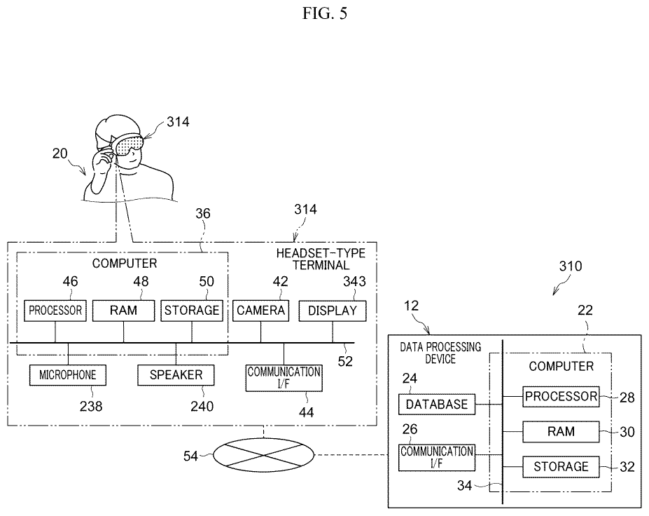

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

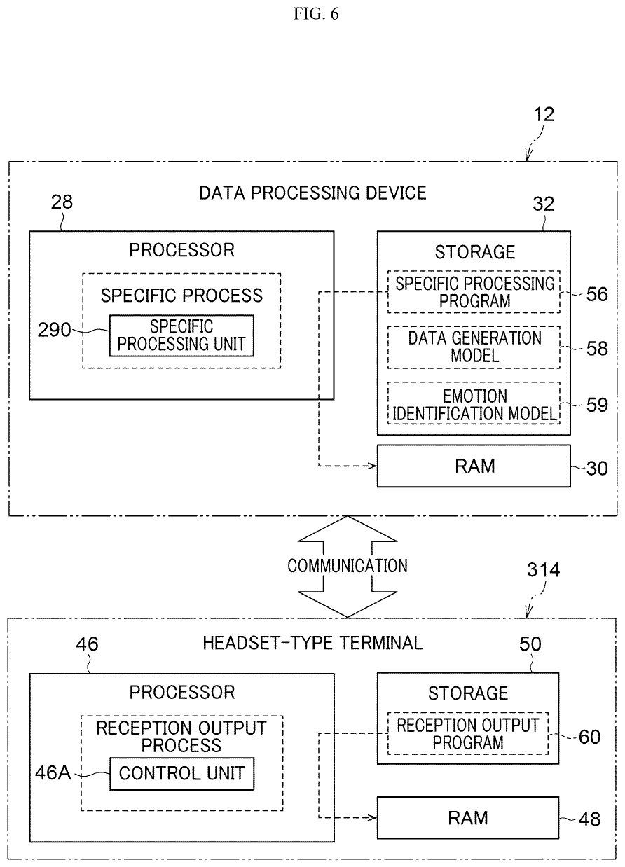

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like.

The data generation model 58 is obtained by performing deep learning with a neural network.

The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### Fourth Exemplary Embodiment

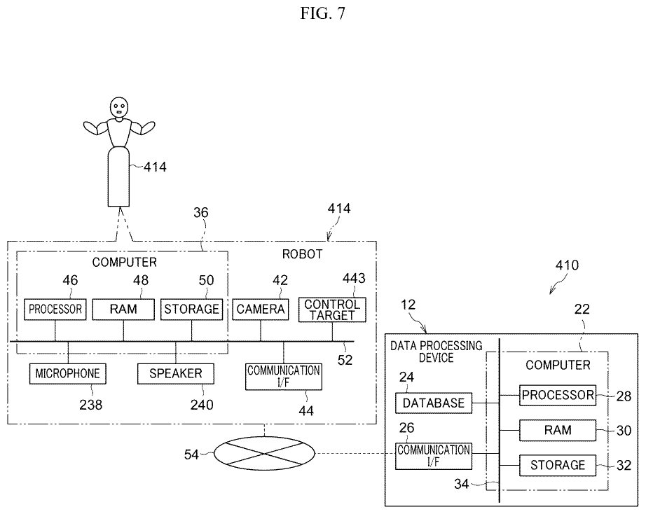

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

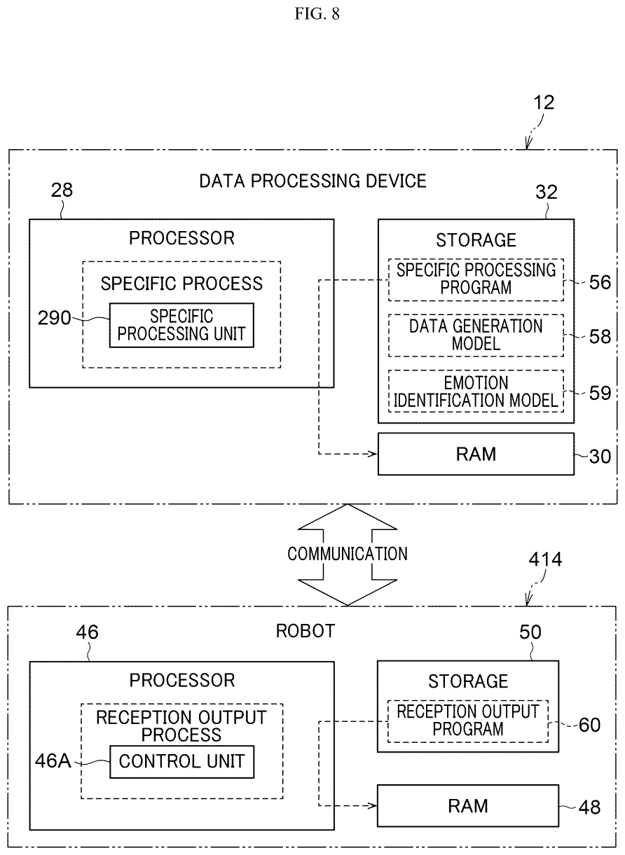

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like.

The data generation model 58 is obtained by performing deep learning with a neural network.

The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

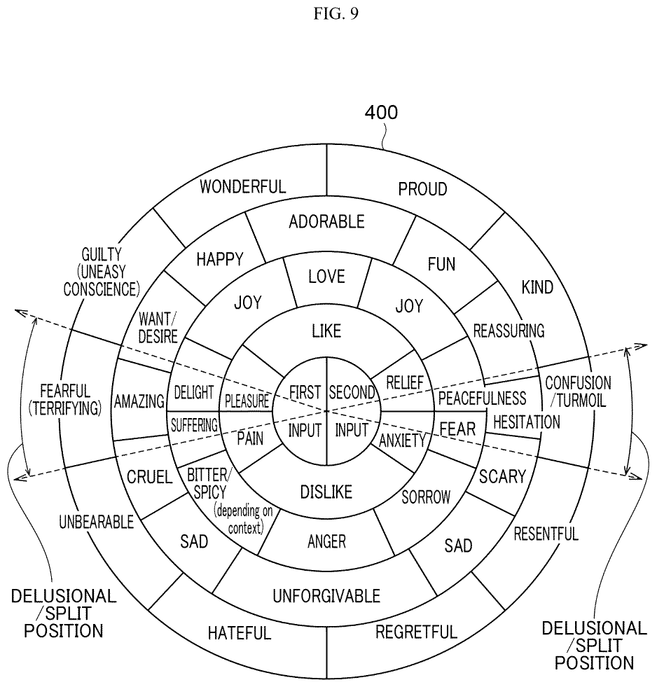

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

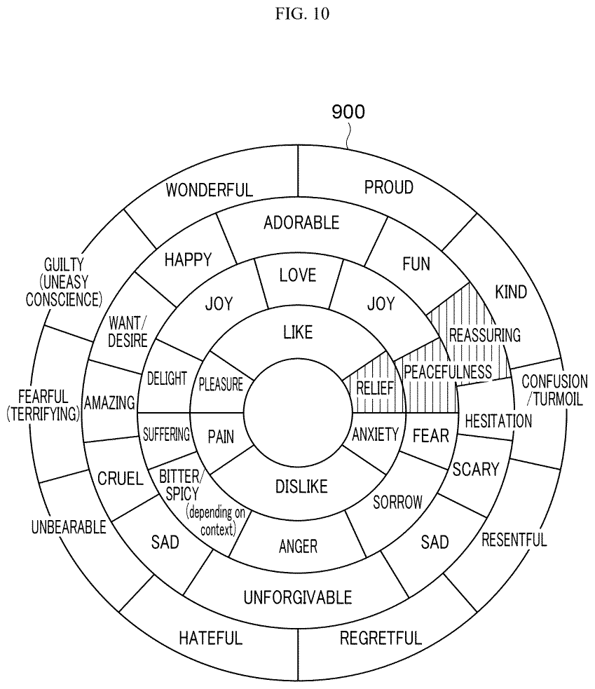

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (SaaS).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### (Supplementary 1)

A system comprising a processor,
wherein the processor is configured to
acquire image data from an image acquisition apparatus that captures an interior of a storage apparatus, and process the image data based on image analysis to specify item information stored in the storage apparatus, and
provide, on a terminal, an information input/output interface through which a user inputs time information for cooking and time information for purchasing items, receive the time information from the terminal via a communication network, and store the time information in a storage device, and
generate structured data including the specified item information and the time information, and generate a prompt sentence in natural language based on the structured data, and input the prompt sentence into a generative AI model, and generate meal plan information including a plurality of cooking menus and an execution time corresponding to each of the cooking menus, based on natural language generation processing performed by the generative AI model, and
convert the generated meal plan information into display data for the terminal and transmit the display data to the terminal via the communication network.

### (Supplementary 2)

The system according to supplementary 1,
wherein the processor is configured to
acquire price information and inventory information of items from an electronic commerce platform via a communication network, generate a prompt sentence for input to the generative AI model based on the acquired item information, the time information, and budget information, obtain, from the generative AI model, meal plan information including candidate purchase items and cooking menus using the candidate purchase items,
automatically select candidate purchase items that conform to the budget information, and transmit purchase processing information regarding the selected candidate purchase items to the electronic commerce platform.

### (Supplementary 3)

The system according to supplementary 1,
wherein the processor is configured to
determine a desired delivery time based on an execution time of the cooking menus included in the meal plan information and the purchase processing information, and transmit delivery request information including the desired delivery time and the candidate purchase items to a delivery management platform so that the candidate purchase items are delivered at a time specified by the user or at a time determined based on the meal plan information.

### Application Example 1

### (Supplementary 1)

A system comprising a processor,
wherein the processor is configured to
acquire image data of a storage apparatus from an image acquisition device, preprocess the image data by using an image analysis algorithm to convert the image data into numerical data, and identify stored objects on a basis of the numerical data to generate stored object information as an identification result, and
cause a terminal device to display a user interface, receive input by a user for time information required for cooking, shopping availability period information, meal provision period information, and meal provision frequency information, convert the input into condition information as structured data, and store the condition information, and
acquire the stored object information and the condition information, integrate the stored object information and the condition information into natural language text, and generate a prompt sentence for input to a generative artificial intelligence model, and
transmit the prompt sentence to the generative artificial intelligence model via a network, receive response text as meal plan information from the generative artificial intelligence model, and analyze the meal plan information to extract dish name information, required stored object information, and cooking procedure information, and
compare the required stored object information with the stored object information to specify shortage stored object information, and convert the meal plan information, the stored object information, and the shortage stored object information into display data capable of being output to the terminal device.

### (Supplementary 2)

The system according to supplementary 1,
wherein the processor is configured to
associate the shortage stored object information with merchandise information, acquire price information and inventory information from an external information providing apparatus on a basis of the merchandise information, automatically generate purchase candidate information on a basis of the price information and budget information, and execute purchase processing via an external transaction processing apparatus on a basis of the purchase candidate information.

### (Supplementary 3)

The system according to supplementary 1,
wherein the processor is configured to
generate delivery request information on a basis of the shortage stored object information and delivery request time information specified by the user, transmit the delivery request information to an external delivery management apparatus to perform delivery processing, acquire delivery result information returned from the external delivery management apparatus, and output the delivery result information to the terminal device.

### Example 2

### (Supplementary 1)

A system comprising a processor,
wherein the processor is configured to
acquire image information indicating a state inside a storage apparatus via an image acquisition device or a portable information terminal, and
acquire, based on the image information and an operation of a user, meal condition information including at least one of a meal execution time, a cooking required time, a number of persons, a nutritional condition, a preference condition, a budget condition, and a cooking time constraint, and
execute analysis processing on the image information by using an image recognition information processing apparatus or an image recognition program, identify ingredient information present inside the storage apparatus, and convert the ingredient information into structured data as standardized ingredient information, and
generate, based on the ingredient information obtained as the structured data and the meal condition information, a prompt sentence for input to a generative AI model by using template information and a character string generation algorithm, and
transmit the prompt sentence via a communication network to an external generative information processing model providing apparatus or an internal generative information processing program, cause the generative AI model to generate meal plan information based on the prompt sentence, and receive response information, and
extract, from the meal plan information, dish name information, step information, and required ingredient information, compare the required ingredient information with the ingredient information of the storage apparatus, calculate shortage ingredient information, and structure display information including the meal plan information and the shortage ingredient information, and
transmit the display information to the portable information terminal, and present, on a user display interface of the portable information terminal, the meal plan information and the shortage ingredient information so that the user can browse, edit, and confirm order candidate information, and
acquire the order candidate information edited or confirmed by the user, and store the order candidate information in association with the shortage ingredient information.

### (Supplementary 2)

The system according to supplementary 1,
wherein the processor is configured to
communicate, based on the shortage ingredient information and the order candidate information edited or confirmed by the user, with a product information management apparatus or an electronic commerce information processing apparatus, generate and transmit order information including product identification information, quantity information, delivery destination information, and desired delivery time information based on the meal condition information, and acquire order confirmation information and scheduled delivery information and notify the portable information terminal of the order confirmation information and the scheduled delivery information.

### (Supplementary 3)

The system according to supplementary 1,
wherein the processor is configured to
in a case where an update request or a re-proposal request for the meal plan information obtained from the generative AI model is received from the portable information terminal, regenerate a new prompt sentence reflecting the ingredient information, the meal condition information, and preference history information of the user, re-input the new prompt sentence to the generative AI model, acquire updated meal plan information, and provide the updated meal plan information to the portable information terminal.

### Application Example 2

### (Supplementary 1)

A system comprising a processor,
wherein the processor is configured to
acquire state information of a storage appliance including an interior of a cooling compartment, and process image data indicating the interior of the storage appliance by using image analysis technology to identify and structure stored ingredient information, and provide, via a user terminal, an information input and output interface through which a user inputs usage conditions including at least one of a meal provision time, a cooking time, a shopping-available time, a menu provision period, a menu provision frequency, budget information, and preference information, and
acquire emotion-related input data or biometric information from which an emotional state of the user is inferable, and determine the emotional state of the user by using emotion analysis technology, and
generate a prompt sentence for input to a generative AI model on the basis of the ingredient information, the usage conditions, and the emotional state, and
transmit the prompt sentence to the generative AI model, and acquire, from the generative AI model, a meal plan including at least a menu name, required ingredients, required quantities, and cooking procedures that satisfy the emotional state and the usage conditions, and compare the required ingredients included in the acquired meal plan with the ingredient information to identify missing ingredients, and
present the meal plan, the missing ingredients, and the cooking procedures to the user terminal, and fix the meal plan in response to a selection operation by the user performed via the user terminal.

### (Supplementary 2)

The system according to supplementary 1,
wherein the processor is configured to
obtain, via a communication network and from a merchandise information providing apparatus, price information and stock information for the missing ingredients, calculate and
propose combinations of candidate ingredients that are purchasable within the budget information, and automatically execute purchase processing for the candidate ingredients based on a selection by the user or a preset condition.

### (Supplementary 3)

The system according to supplementary 1,
wherein the processor is configured to
cooperate with a delivery management apparatus so that, for the candidate ingredients for which the purchase processing has been completed, delivery processing is automatically executed such that the candidate ingredients are delivered at a delivery time designated by the user or at a delivery time corresponding to the meal provision time.

What is claimed is:

1. A system comprising:
circuitry configured to:
acquire, from an image acquisition apparatus coupled to a client terminal via a packet-switched network, image data representing contents of a storage apparatus;
process the image data using an image recognition model to identify items stored in the storage apparatus and generate item information as structured data;
provide, via the packet-switched network to the client terminal, an information input interface through which a user inputs temporal constraint information and resource constraint information;
receive the temporal constraint information and the resource constraint information from the client terminal via the packet-switched network and store the received information in a database;
generate a prompt sentence in natural language based on the item information and the temporal constraint information, the prompt sentence instructing a generative neural network model to generate a plan comprising a plurality of action sequences and an execution time corresponding to each action sequence;
input the prompt sentence to the generative neural network model and obtain response data comprising the plan;
convert the response data into display data and transmit the display data to the client terminal via the packet-switched network;
acquire, from an external data service via the packet-switched network, attribute data and status data for records corresponding to the plan;
select matching records based on the resource constraint information and the attribute data, and generate result data identifying the matching records; and
transmit the result data to the client terminal via the packet-switched network.

2. The system according to claim 1, wherein the circuitry is configured to process the image data by applying an object detection algorithm using a convolutional neural network to extract feature vectors from the image data, classify detected regions into item categories based on the feature vectors, and generate the item information comprising item identifiers and confidence scores for each detected item.

3. The system according to claim 1, wherein the circuitry is configured to generate the structured data by integrating the item information, the temporal constraint information, and the resource constraint information into a data structure, and to generate the prompt sentence by converting the data structure into natural language text comprising descriptions of the identified items and the constraint information.

4. The system according to claim 1, wherein the circuitry is configured to parse the response data from the generative neural network model to extract, for each action sequence, a sequence identifier, required item identifiers, a time duration, and procedural steps, and to store the extracted information in the database.

5. The system according to claim 4, wherein the circuitry is configured to compare the required item identifiers against the item information to determine items that are present in the storage apparatus and items that are absent, and to generate a deficiency list identifying the absent items.

6. The system according to claim 5, wherein the circuitry is configured to transmit the deficiency list to the external data service via the packet-switched network, receive the attribute data and the status data for the absent items, and select the matching records from the absent items based on the resource constraint information.

7. The system according to claim 1, wherein the circuitry is configured to estimate an emotional state of the user based on at least one of text input data and interaction pattern data received from the client terminal, and to adjust a presentation order or content of the plan displayed on the client terminal based on the estimated emotional state.

8. The system according to claim 7, wherein the circuitry is configured to adjust, based on the estimated emotional state, at least one of a complexity level of the action sequences included in the plan and a number of the action sequences presented to the user.

9. The system according to claim 1, wherein the circuitry is configured to generate a second prompt sentence based on the item information, the temporal constraint information, and user preference data stored in the database, the second prompt sentence instructing the generative neural network model to generate an alternative plan that prioritizes items currently stored in the storage apparatus.

10. The system according to claim 1, wherein the circuitry is configured to receive feedback data from the client terminal indicating a user evaluation of the plan, store the feedback data in the database, and incorporate the feedback data into subsequent prompt sentences to improve alignment of generated plans with user preferences.

11. The system according to claim 1, wherein the circuitry is configured to generate the prompt sentence to instruct the generative neural network model to output the plan in a structured format comprising a plurality of fields including action sequence identifiers, required item lists, time allocations, and procedural descriptions, and to parse the response data according to the structured format.

12. The system according to claim 1, wherein the circuitry is configured to acquire the image data at periodic intervals from the image acquisition apparatus, compare current item information with previously stored item information in the database, and generate change detection data indicating items that have been added to or removed from the storage apparatus.

13. The system according to claim 1, wherein the circuitry is configured to receive geographic location information of the user from the client terminal via the packet-switched network, and to preferentially select the matching records from the external data service based on proximity of a distribution source to the geographic location.

14. The system according to claim 1, wherein the storage apparatus is a refrigerator, the items are food ingredients, and the plan comprises a meal plan including cooking menus, and the circuitry is configured to generate the prompt sentence to instruct the generative neural network model to propose the cooking menus based on food ingredients identified in the image data and the temporal constraint information.

15. The system according to claim 14, wherein the circuitry is configured to transmit a query to the external data service via the packet-switched network, the query comprising identifiers of the matching records, quantities, and the resource constraint information, and to receive confirmation data indicating completion of a fulfillment operation.

16. The system according to claim 15, wherein the circuitry is configured to determine a delivery time based on an execution time of a selected action sequence included in the plan, and transmit scheduling request data comprising the delivery time and matching record identifiers to a logistics service via the packet-switched network.

17. The system according to claim 1, wherein the circuitry is configured to generate nutritional analysis data by analyzing the plan against a nutritional reference database stored in the database, and to transmit the nutritional analysis data to the client terminal for display alongside the plan.

18. A system comprising:
circuitry configured to:
acquire, from an image acquisition apparatus coupled to a client terminal via a packet-switched network, image data representing contents of a storage apparatus;
process the image data using an image recognition model to identify items and generate item information;
receive temporal constraint information and resource constraint information from the client terminal via the packet-switched network;
generate a prompt sentence based on the item information and the constraint information, the prompt sentence instructing a generative neural network model to generate a plan;
input the prompt sentence to the generative neural network model and obtain response data;
acquire, from an external data service via the packet-switched network, attribute data and status data for records corresponding to the plan;
select matching records based on the resource constraint information;
estimate an emotional state of the user based on interaction data received from the client terminal;
adjust at least one of a content of the plan and a selection of the matching records based on the estimated emotional state; and
transmit the plan and the result data to the client terminal via the packet-switched network.

19. The system according to claim 18, wherein the circuitry is configured to receive selection data from the client terminal indicating a user-selected plan and user-approved matching records, and to transmit a fulfillment request to the external data service via the packet-switched network based on the selection data.

20. A method performed by circuitry, the method comprising:
acquiring, from an image acquisition apparatus coupled to a client terminal via a packet-switched network, image data representing contents of a storage apparatus;
processing the image data using an image recognition model to identify items and generate item information;
receiving temporal constraint information and resource constraint information from the client terminal via the packet-switched network;
generating a prompt sentence based on the item information and the constraint information, the prompt sentence instructing a generative neural network model to generate a plan;
inputting the prompt sentence to the generative neural network model and obtaining response data;
acquiring, from an external data service via the packet-switched network, attribute data and status data for records corresponding to the plan;
selecting matching records based on the resource constraint information; and
transmitting the plan and result data identifying the matching records to the client terminal via the packet-switched network.
